(19) United States
(12) Patent Application Publication
(10) Pub. No.: US 2026/0076878 A1
(43) Pub. Date: Mar. 19, 2026

(54) ORAL LIQUID DENTAL SUSPENSION FORMING A VARNISH FOR CONTROLLED TEETH WHITENING AND ENAMEL CARE

(71) Applicant: Gary J. Speier, Minneapolis, MN (US)

(72) Inventor: Gary J. Speier, Minneapolis, MN (US)

(21) Appl. No.: 19/326,845

(22) Filed: Sep. 12, 2025

Related U.S. Application Data
(60) Provisional application No. 63/696,067, filed on Sep. 18, 2024.

(51) Int. Cl. A61K 6/40; A61K 6/20; A61K 6/60; A61K 6/70; A61K 8/22; A61Q 11/02

(52) U.S. Cl. CPC A61K 6/40; A61K 6/20; A61K 6/60; A61K 6/70; A61K 8/22; A61Q 11/02

(57) ABSTRACT

An oral liquid dental suspension is disclosed, including (a) a tooth whitening agent; (b) a solvent; and (c) at least one of (i) a resin or (ii) a urethane. The suspension is formulated to be applied to the surface of human teeth, where it rapidly dries—even in the presence of saliva—to form an adherent varnish. This varnish enables prolonged and effective contact between the whitening agent and tooth enamel for enhanced whitening efficacy. The composition may further include one or more optional additives, such as sweeteners, glazing agents, teeth desensitizers, remineralization agents, sequestering agents, complexing agents, or fluoridation agents. Also provided are kits containing the oral liquid dental suspension and one or more applicators, as well as methods of use involving topical application to teeth and subsequent drying to achieve a whitening effect.

### BACKGROUND

Tooth discoloration and staining are common dental concerns affecting individuals of all ages. Discoloration can arise from a variety of intrinsic and extrinsic sources, including the consumption of coffee, tea, wine, tobacco products, certain foods, aging, dental trauma, and exposure to tetracycline antibiotics or excessive fluoride. As a result, there is significant demand for effective and convenient tooth whitening products and procedures.

Numerous methods for whitening teeth have been developed, ranging from professional in-office treatments, such as high-concentration hydrogen peroxide or carbamide peroxide bleaching gels, to over-the-counter products including whitening toothpastes, rinses, strips, trays, and paint-on gels. In-office whitening procedures often require the use of trays or barriers to protect soft tissues and sometimes incorporate specialized light sources (UV, halogen, LED, plasma arc) or heat to accelerate bleaching. These approaches can deliver rapid results but typically involve higher concentrations of bleaching agents, which may cause tooth sensitivity, gingival irritation, and risks associated with accidental ingestion. The need for professional supervision, specialized equipment, or customized trays may also limit patient access and convenience.

Conventional over-the-counter whitening products generally contain lower concentrations of bleaching agents and often rely on repeated, prolonged application (such as gels in trays worn overnight or strips applied for 30-60 minutes daily over several weeks) to achieve noticeable whitening. While more accessible, these methods often suffer from variable efficacy, uneven exposure of active agents to tooth surfaces, difficulties in application, and unwanted side effects like heightened tooth sensitivity or irritation to soft oral tissues. Paint-on gels and similar products may lack sufficient adhesion, leading to premature loss due to saliva or oral movements, which reduces efficacy, decreases consistent bleaching time, and increases the risk of inadvertent swallowing of active agents. In addition, many available whitening compositions provide little or no benefit for teeth with existing dental work such as caps, crowns, implants, bridges, bonded teeth, and veneers. The harsh or abrasive nature of some preparations may damage enamel, promote demineralization, or erode dental restorations. Sensitivity, taste, ease of use, safety for adolescents, and the ability to selectively treat individual or multiple teeth remain persistent challenges. Furthermore, most systems are not designed to incorporate desensitizing or remineralizing agents, nor do they provide methods for extended contact or controlled release of active ingredients.

Accordingly, there remains a need for improved teeth whitening compositions and methods that combine rapid setting, strong adhesion, prolonged contact with tooth surfaces, and the option to include desensitizing and remineralizing components. Such systems should ideally minimize irritation, ingestion, and the effects of saliva while being easily applicable to all or selected teeth, with or without a gag reflex, and suitable for both professional and home use. Additionally, the ability to use such systems in conjunction with light or heat sources to enhance whitening, or alongside pre- and post-treatment rinses and agents, would provide further advantages over the current state of the art.

### SUMMARY

The present invention provides for an oral liquid dental suspension that includes: (a) tooth whitening agent; (b) solvent; and (c) at least one of (i) resin and (ii) urethane. The oral liquid dental suspension may optionally further include one or more additional substances (e.g., sweetener, flavoring agent, glazing agent, teeth desensitizer, remineralization agent, sequestering agent, complexing agent, bone regeneration agent, and/or fluoridation agent). Upon application to the surface of human teeth, the oral liquid dental suspension dries quickly, in the presence of saliva, to form a varnish. The sticky consistency of the oral liquid dental suspension helps it to adhere to the tooth's surface, thereby allowing the tooth whitening agent to remain in contact with the tooth for an extended period of time after the suspension dries to form a varnish (e.g., up to several hours). Over the extended period of time, the tooth whitening agent penetrates into the teeth and breaks up the discolorations on the inner aspects of the teeth, thereby whitening them. Repeated treatments with the oral liquid dental suspension can improve the shade of the teeth multiple VITA® Bleachedguide shades.

The present invention also provides for a method (e.g., a method of medical treatment, a method of dental treatment, and/or a method of cosmetic procedure). The method includes applying the oral liquid dental suspension to the surface of human teeth (e.g., buccal/labial surfaces of the human teeth) and allowing the suspension to dry to form a varnish. The method includes, e.g., a method of teeth whitening, a method of lightening the color of human teeth, and/or a method of bleaching human teeth.

The present invention also provides for a kit that includes the oral liquid dental suspension and an applicator (e.g., applicator brush). The oral liquid dental suspension can be contained, e.g., in a tube (e.g., sealed dentinal tube), unidose package, syringe dispenser, vial, or pen applicator. The kit may optionally further include another oral liquid solution, to be used pre-bleaching and/or post-bleaching, that includes a teeth desensitizer or remineralization agent. The kit may optionally further include another oral liquid solution, to be used post-bleaching, for fluoridation. The kit may optionally further include teeth whitening equipment that provides for a source of light energy (e.g., UV, halogen, LED, or plasma arc) and/or heat, for use in the method of medical treatment, dental treatment, and/or cosmetic procedure. The kit can further include an oral liquid solution containing a bleach activator that includes tetraacetylethylenediamine (TAED), sodium nonanoyloxybenzenesulfonate (NOBS), or a combination thereof.

Across multiple embodiments, the oral liquid dental suspension, and the method of use described herein, can independently possess one or more advantages relative to other teeth whitening compositions (e.g., gels, strips, etc.). The advantages can relate to any one or more of the following.
The oral liquid dental suspension can dry rapidly to form a varnish.
The oral liquid dental suspension can set (to form a varnish) in the presence of saliva.
Because the oral liquid dental suspension does not require the use of trays, it is suitable for use in patients with a strong gag reflex.
The oral liquid dental suspension can be applied to all teeth, selective teeth, or to a single tooth.
During the method described herein, the oral liquid dental suspension can be re-applied, one or more times, to all teeth, selective teeth, or to a single tooth.
Due to the amounts used and the rapid setting time of the oral liquid dental suspension, a small or negligible amount of tooth whitening agent is typically ingested.
Due to the rapid setting time of the oral liquid dental suspension, a small or negligible amount of tooth whitening agent is typically washed away by saliva.
The oral liquid dental suspension has a sticky consistency, which helps it to adhere to the tooth's surface, thereby mitigating any issues with saliva buildup during the teeth-whitening session.
The oral liquid dental suspension has a sticky consistency, which helps it to adhere to the tooth's surface, thereby allowing the tooth whitening agent to remain in contact with the tooth for an extended period of time (e.g., up to several hours) after the suspension dries to form a varnish.
The oral liquid dental suspension can be applied relatively easily and quickly.
The oral liquid dental suspension can be applied by the consumer (e.g., at home), by a dental professional (e.g., in-office treatment), or esthetician (e.g., in-office treatment).
The oral liquid dental suspension can be applied and used, with or without a teeth whitening system that provides light energy (e.g., UV, halogen, LED, and/or plasma arc) and/or heat to catalyze the tooth whitening agent.
The oral liquid dental suspension can be applied and used to form a varnish, while providing a temporary protective barrier to the teeth, thereby allowing the patient to consume liquid beverages (e.g., coffee, tea, wine, soda, etc.) without concern of staining the teeth.
The oral liquid dental suspension can be applied and used to form a varnish on select portions of the teeth (e.g., buccal/labial surfaces), without exposing to the tooth whitening agent other teeth, other surfaces of the teeth, or the gums.
The oral liquid dental suspension is safe for enamel and produces minimal sensitivity, irritation, or pain.
The oral liquid dental suspension is safe for enamel, caps, crowns, implants, bridges, bonded teeth, and veneers.
The oral liquid dental suspension can set to form a varnish that is discrete and convenient for use while in public.
When the oral liquid dental suspension is applied to the teeth, the tooth whitening agent penetrates into the tooth enamel and provides extended (e.g., 0.1-12 hours) whitening of the teeth.
The oral liquid dental suspension can set to form a varnish, which adheres to the teeth, creating a coating for treatment over an extended period of time (e.g., 0.1-12 hours).
Upon application to the surface of human teeth, the oral liquid dental suspension dries quickly to form a varnish, while maintaining the tooth whitening agent in direct contact with the teeth throughout the teeth whitening process (e.g., 0.1-12 hours).
When the oral liquid dental suspension is applied to the teeth, the tooth whitening agent penetrates into the teeth and breaks up the discolorations on the inner aspects of the tooth, thereby whitening them.
The oral liquid dental suspension provides a desensitizing action when applied to enamel and dentin surfaces.
The oral liquid dental suspension helps to remineralize teeth and decreases tooth hypersensitivity.
The oral liquid dental suspension effectively causes oxidation on teeth, breaking down surface stains and reducing their hue. The breakdown of these stains results in a whiter smile without damage to the teeth or gums.
The oral liquid dental suspension is safe to use on sensitive teeth.
While it varies based on the frequency of touch-ups, oral hygiene practices, and lifestyle habits of the individual, whitening results can last 6 months to 2 years.
The oral liquid dental suspension can be effective on both extrinsic and intrinsic staining.
Fewer occurrences and/or less severity of common adverse effects of vital tooth whitening (e.g., temporary tooth sensitivity and gingival inflammation).
The oral liquid dental suspension can be formulated as an over-the-counter (OTC) product, for home use by the consumer, as well as a prescription (Rx) grade product, for in-office use to be applied by a dental professional, esthetician, etc.
The oral liquid dental suspension can be formulated to form a varnish that adheres to the teeth, creating a coating for treatment, that stays essentially intact over a desired and targeted period of time, e.g., from 0.1 hours to 12 hours.
The oral liquid dental suspension can be formulated to include substances currently in use with commercially available oral dental products.
The oral liquid dental suspension can be formulated to mitigate tooth sensitivity.
The oral liquid dental suspension can be formulated to mitigate enamel demineralization.
The oral liquid dental suspension can be formulated to mitigate enamel erosion.
The oral liquid dental suspension can be formulated to mitigate reduction in surface microhardness of the enamel.
Repeated treatments with the oral liquid dental suspension can improve the shade of the teeth multiple VITA® Bleachedguide shades.

The present invention therefore addresses existing shortcomings of conventional teeth whitening techniques by providing an oral liquid dental suspension that forms a rapidly-setting varnish, ensuring extended contact of whitening agents and optional therapeutic components with the tooth surface for improved efficacy, safety, and convenience.

### DETAILED DESCRIPTION

The present invention can be more readily understood by reading the following detailed description of the invention and study of the included examples.

As used herein, the following terms have the meanings ascribed to them unless specified otherwise.

The terms “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The term “about” modifies the subject values to indicate a margin of error typically within ±10% unless otherwise specified.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “weight percent” (sometimes written as wt. % or % w/w) expresses the concentration of a component in a mixture as the number of grams of that component per 100 grams of the total mixture.

### Materials, Ingredients, and Components

The term “liquid suspension” refers to a flowable composition or mixture wherein solid, liquid, or semi-solid components are dispersed or suspended in a continuous liquid phase-such as a solution, emulsion, or colloidal system-suitable for application to tooth surfaces via spreading, brushing, spraying, or painting.

The term “two-part oral liquid dental suspension” refers to a dental whitening product system that is separated into two distinct liquid components, each kept isolated until the moment of use. The first part typically contains the tooth whitening agent (which may be peroxide-based or non-peroxide-based) in a liquid vehicle (such as ethanol, water, or propylene glycol). The second part typically contains the film-forming resin (such as polyvinyl acetate, shellac, hydrogenated rosin, or synthetic resin) and usually also in a liquid carrier. The two parts are mixed together right before application to teeth. This is often done using a dual-syringe, dual-chamber pen, ampoule, or other kit delivery mechanism. The separation improves chemical stability (especially for unstable peroxide whitening agents) and preserves the performance of both the active whitening agent and film-forming base until use. After mixing, the resulting homogeneous suspension is applied to the teeth and quickly dries to form a varnish layer for sustained dental agent contact.

The term “varnish” refers to the film or layer formed in situ on teeth after the dental suspension dries, sets, cures, or crosslinks; this film adheres to the teeth surfaces, providing extended retention and controlled delivery of active ingredients such as whitening agents, remineralizers, or desensitizers.

The term “tooth whitening agent” or “teeth whitening agent” refers to any chemical, compound, or substance—such as hydrogen peroxide, carbamide peroxide, phthalimidoperoxycaproic acid (PAP), or other peroxides or oxidizing agents—capable of promoting, facilitating, or effecting the removal, bleaching, oxidation, reduction, or masking of stains or discoloration, whether intrinsic or extrinsic, on teeth.

The term “solvent” refers to any liquid or mixture (organic or inorganic, hydrophilic or hydrophobic, single or multi-component) that dissolves, dilutes, or carries other ingredients in the composition and facilitates application, spreading, or drying; examples include ethanol, glycerin, propylene glycol, and water.

The term “resin” refers to any natural, synthetic, semi-synthetic, or modified polymeric or oligomeric material-including, but not limited to, colophony (rosin), shellac, mastic, synthetic methacrylate resins, or polyvinyl acetate-capable of providing adhesive, film-forming, thickening, or setting properties to the dental varnish.

The term “urethane” refers to any organic compound containing one or more urethane (carbamate) linkages or derivatives (including urethane dimethacrylate), existing as monomers, oligomers, or polymers, and used in dental varnishes for their film-forming, adhesive, and structural properties.

The term “phthalimidoperoxycaproic acid” or “PAP” refers to a synthetic, organic peracid compound, including all isomers, salts, hydrates, and derivatives thereof, capable of acting as an oxidizing agent suitable for tooth whitening.

The term “teeth desensitizer” refers to any agent or compound that reduces the sensation of pain, sensitivity, or discomfort associated with teeth, including potassium nitrate, potassium citrate, calcium phosphates, tricalcium phosphate, tetrapotassium pyrophosphate, or hydroxyapatite.

The term “remineralization agent” refers to any material or formulation that provides, enhances, or promotes the deposition of minerals such as calcium or phosphate ions to enamel or dentin, strengthening or repairing tooth structure; examples include hydroxyapatite, nano-hydroxyapatite, and tricalcium phosphate.

The term “peroxide” refers to any compound, salt, hydrate, or composition containing an oxygen-oxygen single bond (peroxide bond), including organic and inorganic peroxides, capable of releasing active oxygen or exhibiting oxidative bleaching or disinfecting properties.

The term “hydrogen peroxide” refers to the inorganic peroxide H₂O₂, in any concentration, in pure, diluted, or formulated form, capable of acting as an oxidizer and tooth whitening agent.

The term “carbamide peroxide” refers to the urea-hydrogen peroxide adduct, including all commercially available grades, which upon decomposition provides hydrogen peroxide and urea for tooth whitening purposes.

The term “sodium perborate” refers to any hydrated or anhydrous salt of perboric acid with sodium, used as an oxidant or bleaching agent.

The term “sodium percarbonate” refers to any sodium salt of percarbonate (sodium carbonate peroxide), typically used as a source of hydrogen peroxide and for bleaching purposes.

The term “sodium persulfate” refers to any sodium salt of peroxydisulfuric acid, including sodium peroxydisulfate, employed as an oxidizing or bleaching agent.

The term “calcium peroxide” refers to any composition containing calcium and peroxide species, capable of delivering oxygen and suitable for use in dental bleaching or cleaning.

The term “magnesium peroxide” refers to magnesium peroxide compounds capable of liberating oxygen and effecting bleaching or whitening.

The term “urea peroxide” refers to a hydrogen peroxide-liberating compound (distinct from carbamide peroxide) with urea linkage, suitable for dental bleaching.

The term “zinc peroxide” refers to a class of zinc-containing peroxides, in anhydrous or hydrated form, capable of functioning as an oxidizing or bleaching agent.

The term “ethanol” refers to ethyl alcohol (C₂H₅OH), including all grades and concentrations, used for its solvent, carrier, or drying properties.

The term “glycerin” refers to glycerol (1,2,3-propanetriol) and its derivatives, in anhydrous or hydrated form, used as a humectant, solvent, or viscosity modifier.

The term “propylene glycol” refers to 1,2-propanediol in any grade, used as a solvent, plasticizer, or humectant.

The term “xylitol” refers to the five-carbon sugar alcohol (pentitol) used as a non-cariogenic sweetener.

The term “sucralose” refers to the chlorinated sucrose-based artificial sweetener, in any grade or concentration.

The term “sorbitol” refers to the hexitol sugar alcohol derived from glucose, used as a sweetener, humectant, or plasticizer.

The term “sodium saccharin” refers to the sodium salt of saccharin, an artificial non-nutritive sweetener.

The term “flavoring agent” refers to any natural, synthetic, or artificial substance or mixture added to modify, enhance, or mask the taste or aroma of the product, including flavors like peppermint oil, menthol, eucalyptol, thymol, fruit flavors, caramel, or bubblegum.

The term “caramel” refers to the flavoring and/or coloring agent derived from heat-treated sugars, including natural or artificial forms.

The term “menthol” refers to the cyclic monoterpene alcohol, occurring naturally or synthetically, imparting a mint taste and cooling sensation.

The term “peppermint oil” refers to any essential oil or extract derived from Mentha piperita, containing principal flavor components such as menthol and menthone.

The term “spearmint” refers to natural or synthetic flavor derived from or mimicking the flavor profile of Mentha spicata.

The term “mint” refers to any flavor or aromatic component characteristic of plants of the Mentha genus or formulations replicating the same.

The term “eucalyptol” refers to the cyclic ether also known as 1,8-cineole, found in Eucalyptus species and used for flavoring or aromatic purposes.

The term “thymol” refers to the aromatic monoterpene phenol derived from thyme or other sources, used for flavor or antimicrobial properties.

The term “bubblegum flavor” refers to any composite flavoring reminiscent of commercially available bubblegum products, composed of natural or artificial ingredients.

The term “fruit flavor” refers to flavoring substances or combinations that evoke or mimic the sensory properties of fruit(s), whether natural or synthetic in origin.

The term “colophony (rosin)” refers to a natural resin obtained from pine tree exudate or other sources, potentially modified by hydrogenation or esterification.

The term “mastic” refers to the natural resin secreted from Pistacia lentiscus or synthetic analogs used for film-forming and adhesive properties.

The term “hydrogenated rosin” refers to colophony (rosin) that has been chemically reduced by hydrogenation, altering its physical and chemical properties.

The term “hydrogenated resin” refers to any resin (e.g., natural or synthetic) that has undergone hydrogenation to improve stability, clarity, or performance.

The term “modified rosin” refers to chemically altered forms of natural rosin, including but not limited to those that are esterified, polymerized, or otherwise derivatized to alter solubility or reactivity.

The term “synthetic resins” (CAS No. 68990-03-3) refers to polymeric resins identified by CAS No. 68990-03-3 and any equivalents or blends used for their film-forming, adhesive, or rheological properties.

The term “methyl esters of hydrogenated rosin” (CAS No. 8050-15-5) refers to esterified derivatives of hydrogenated rosin, possessing CAS No. 8050-15-5, and their functional equivalents.

The term “glazing agent” refers to any natural, synthetic, or semi-synthetic substance—such as white wax (E901), carnauba wax, candelilla wax, microcrystalline wax, shellac, or beeswax—that imparts a glossy, smooth, protective coating on teeth or on the applied varnish.

The term “white wax (E901)” refers to bleached beeswax, natural or refined, or equivalent substances used as food-grade or pharmaceutical glazing agents.

The term “stearic acid (E570)” refers to any grade or purified mixture of saturated fatty acids dominated by stearic acid, typically used as a lubricant, stabilizer, or glazing agent.

The term “beeswax (E901)” refers to the natural secreted wax of honeybees, purified or modified for use in compositions, including as a glazing or film-forming agent.

The term “candelilla wax (E902)” refers to the wax obtained from Euphorbia antisyphilitica or related species, or functional synthetic equivalents, used primarily as a glazing or structuring agent.

The term “carnauba wax (E903)” refers to the waxed esters or mixed esters derived from the leaves of Copernicia prunifera or functional analogs, typically providing a hard, shiny finish.

The term “shellac (E904)” refers to the natural resin secreted by the lac bug (Kerria lacca), either unmodified or partially modified, and used as a glazing or surface-sealing agent.

The term “microcrystalline wax (E905c)” refers to a complex mixture of saturated, branched, or cyclic hydrocarbons refined from petroleum, or their synthetic equivalents, with fine crystallinity and used for texture or coating.

The term “crystalline wax (E907)” refers to high-melting, crystalline hydrocarbon waxes of mineral or synthetic origin.

The term “lanolin (E913)” refers to the purified wool wax, fat, or related compounds isolated from sheep's wool, or functionally equivalent synthetic analogs.

The term “oxidized polyethylene wax (E914)” refers to polyethylene wax chemically processed by oxidation, used as a lubricant or glazing agent.

The term “esters of colophonium (E915)” refers to esters (e.g., ethyl, methyl, or glycerol esters) formed from colophony, used to modify solubility, adherence, or rheology.

The term “paraffin” refers to any long-chain hydrocarbon wax derived from petroleum, coal, or shale oil, or synthetic analogs suitable for use as a glazing or structural agent.

The term “potassium nitrate” refers to the inorganic salt KNO₃, used for its desensitizing or other therapeutic actions in oral care products.

The term “potassium citrate” refers to the potassium salt of citric acid, used as a buffering, desensitizing, or remineralizing agent.

The term “tricalcium phosphate” refers to the calcium phosphate salt Ca₃(PO₄)₂, used for remineralization or as a bioactive component.

The term “tetrapotassium pyrophosphate” refers to the potassium salt K₄P₂O₇, used as a sequestering, buffering, or desensitizing agent.

The term “hydroxyapatite” refers to any natural or synthetic calcium phosphate mineral approximating Ca₅(PO₄)₃(OH), including all particle sizes, used for tooth repair, remineralization, or cosmetic benefits.

The term “nano-hydroxyapatite” refers to hydroxyapatite particles predominantly in the sub-100 nanometer range, as well as formulations with a mean particle size below 1 micron, utilized for effective penetration or repair of dental surfaces.

The term “bone regeneration agent” refers to compounds, materials, or compositions intended to support bone growth, repair, remineralization, or regeneration, including but not limited to calcium-based salts or phosphates.

The term “calcium sulfate dihydrate” refers to the inorganic salt CaSO₄·2H₂O, used for bone regeneration or remineralization purposes.

The term “dibasic sodium phosphate” refers to Na₂HPO₄ or related hydrated forms used for buffering, remineralization, or as a mineral source.

The term “ethylenediaminetetraacetic acid (EDTA)” refers to the polyaminocarboxylic acid chelator and its salts, used to sequester metal ions, stabilize peroxides, or prevent discoloration.

The term “complexing agent” refers to any substance (such as cyclodextrin) capable of reversibly or irreversibly binding to other chemical species, ions, or molecules, thereby modifying the stability, solubility, or reactivity of actives (e.g., for enhanced delivery or stabilization).

The term “cyclodextrin” refers to cyclic oligosaccharides (e.g., α-, β-, γ-cyclodextrin) or their derivatives, functioning as inclusion or complexation agents to stabilize or modulate active components.

The term “fluoridation agent” refers to any chemical or formulation providing a source of bioavailable fluoride ions to benefit dental health or support remineralization, including sodium fluoride, stannous fluoride, difluorosilane, hexafluorosilicic acid, and hexafluorosilicate.

The term “sodium fluoride” refers to NaF in any hydrated or anhydrous form, used as the primary fluoride source.

The term “difluorosilan” refers to difluorosilane or analogous fluorinated silanes, if present, for dental fluoridation or protection.

The term “stannous fluoride” refers to SnF₂, used as an antimicrobial or remineralizing fluoride compound.

The term “hexafluorosilicic acid” refers to H₂SiF₆, a source of fluoride used in certain formulations.

The term “hexafluorosilicate” refers to salts containing the anion SiF₆²⁻, used for fluoridation or remineralization.

The term “polymeric matrix” refers to a solid or semi-solid network, often created by the film-forming resins or synthetic polymers (such as shellac, polyvinyl acetate, methacrylate polymers, or similar substances), in which other active ingredients (like whitening agents, remineralizers, or desensitizers) are dispersed, embedded, or encapsulated. The matrix provides structural integrity and controls how these actives are released or retained on the tooth surface over time.

The term “non-drip” refers to the oral liquid dental suspension is engineered and formulated so that it does not run or drip off the applicator or teeth during application. This property is primarily achieved through optimizing the viscosity and rheology of the varnish, ensuring that when applied, the product remains in place and does not form droplets that roll off or create messes on teeth, gums, or surrounding oral tissues.

The term “string-free” refers to the absence of thread-like filaments or “strings” that can form between the applicator (such as a brush or pen) and the treated tooth surface when the varnish is dispensed or applied. A string-free formulation ensures a clean break, allowing the product to be painted on smoothly and precisely without stretchy, sticky residues that could impair accurate placement or lead to waste and user frustration.

The term “clump-free” refers to a varnish that is smooth and homogeneous in texture, meaning it does not form lumps, aggregates, or clots during storage, mixing, or application. A clump-free suspension provides for uniform application and coverage, preventing uneven distribution of active ingredients and ensuring aesthetic and functional performance on each tooth.

The term “continuous film” refers to a single, unbroken, and uniform layer of varnish/coating that forms on the tooth surface after the dental suspension is applied and allowed to dry or set. This film covers the targeted tooth area completely, without holes, breaks, or thin spots, ensuring consistent protection, retention, and sustained release of the whitening or therapeutic agents. The continuous nature of the film enhances tooth contact time, minimizes wash-off by saliva, and supports the controlled delivery profile described in the patent.

The term “animal-derived ingredients” refers to substances that are obtained, extracted, or processed from any animal source, either directly or indirectly. This includes ingredients such as beeswax, shellac, lanolin (from sheep's wool), gelatin (from animal collagen), milk derivatives, honey, carmine (from insects), or any other material that originates from, is produced by, or is a byproduct of animals. The patent specifically mentions vegan and cruelty-free formulations, indicating that in certain embodiments, the dental varnish is formulated to be free from all animal-derived ingredients to accommodate users with dietary, medical, or ethical restrictions.

The term “sodium lauryl sulfate” or “SLS” refers to a widely used synthetic surfactant and cleansing agent with the chemical formula CH₃(CH₂)₁₁OSO₃Na. In oral care products, SLS functions as a detergent and foaming agent that helps emulsify and remove debris. The patent mentions SLS-free embodiments, reflecting some patients' needs for formulations without this ingredient-typically due to concerns about oral tissue irritation or allergies. SLS is a common ingredient in conventional toothpastes, but is excluded from some formulations to make the dental varnish suitable for sensitive populations.

The term “encapsulated agent” refers to an active ingredient embedded or protected within microcapsules, matrices, or similar technologies for controlled or sustained release on tooth surfaces.

The term “sweetener” refers to any substance, synthetic or naturally derived, used to impart a sweet taste, including sugars (e.g., xylitol, sorbitol), sugar alcohols, and non-nutritive sweeteners such as sucralose and sodium saccharin.

The term “color-change indicator” refers to an additive—such as a dye, pigment, or UV-fluorescent tracer (e.g., pH-sensitive dyes, FD&C Blue #1, titanium dioxide, mica, fluorescein)—incorporated to provide a temporary visible signal during application, becoming clear, undetectable, or visually insignificant on teeth after the varnish sets.

The term “stabilizer” refers to any substance (such as EDTA or antioxidants) added to the varnish composition to increase storage stability and shelf life by preventing premature breakdown, degradation, or discoloration of active ingredients.

The term “sequestering agent” refers to any compound (such as ethylenediaminetetraacetic acid, EDTA) that binds, chelates, or sequesters metal ions to stabilize active ingredients or inhibit undesirable chemical reactions within the formulation.

The term “bleach activator” refers to a substance that improves bleaching agent activity; examples include TAED (tetraacetylethylenediamine) and NOBS (sodium nonanoyloxybenzenesulfonate).

The term “tetraacetylethylenediamine” or “TAED” refers to TAED in any form, used as a bleach activator or stain remover in oral compositions.

The term “sodium nonanoyloxybenzenesulfonate” or “NOBS” refers to the sodium salt of nonanoyloxybenzenesulfonic acid, functioning as a bleach or whitening booster.

The term “anatase” refers to a tetragonal crystalline polymorph of titanium dioxide (TiO₂) used as a pigment, catalyst, or additive in nano- or bulk form.

The term “rutile” refers to a tetragonal crystalline polymorph of titanium dioxide (TiO₂) used as a pigment, catalyst, or additive in nano- or bulk form.

The term “brookite” refers to an orthorhombic crystalline polymorph of titanium dioxide (TiO₂), used as a pigment, catalyst, or additive in nano- or bulk form.

The term “prophy paste” refers to prophylactic dental abrasive paste, typically containing polishing agents, used during professional cleaning.

The term “titanium oxide nanoparticle-based catalyst” refers to any formulation containing titanium dioxide nanoparticles (anatase, rutile, or brookite phase) used to catalyze bleaching, decompose whitening agents, or afford enhanced cleaning.

### Anatomy, Application, Devices, and Clinical Terms

The term “intervening treatment” refers to any dental, medical, cosmetic, or cleaning procedure, process, or substance applied to the teeth or mouth that occurs between the sequential steps outlined in the claimed method-specifically, for example, between step (1) (removing biofilm/extrinsic staining), step (2) (rinsing with a bleach activator solution), step (3) (application and drying of the dental suspension/varnish), and step (4) (light exposure). In specific embodiments, the method may specify that these steps be performed in direct sequence, without any additional procedure or product use in between that could alter or affect the condition of the teeth, the efficacy of the whitening system, or the intended performance of the dental varnish during the process.

The term “surface of human teeth” refers to any exposed or accessible part of a human tooth, including enamel, dentin, or restorative surfaces, regardless of tooth type, location, or underlying condition.

The term “buccal/labial surfaces of human teeth” refers to the outer-facing tooth surfaces, namely the surfaces of anterior (labial) and posterior (buccal) teeth that face toward the lips or cheeks.

The term “teeth whitening” (as well as related expressions such as “bleaching” and “lightening the color of human teeth”) refers to any chemical, physical, or biological process, treatment, or method that results in a reduction of tooth discoloration or an increase in the perceptible whiteness or brightness of teeth. The distinction between “whitening” and “bleaching” is established by context: “whitening” broadly encompasses the removal or oxidation of extrinsic and intrinsic stains and is often used interchangeably with “bleaching,” although it can include any effect that enhances tooth brightness, not solely chemical bleaching. The term “lightening” specifically refers to those processes, products, or actions that reduce stains, increase reflectance, or move tooth shade toward a whiter value.

The term “saliva” refers to the natural or simulated oral fluid found in the mouth of a human, containing water, ions, proteins, and other constituents and capable of affecting composition setting, taste, or dissolution.

The term “gag reflex” refers to the normal or exaggerated involuntary contraction response elicited by stimuli within the mouth or oropharynx, which may complicate or limit oral care procedures.

The term “patient” refers broadly to any recipient, user, or subject to whom the dental varnish or treatment is applied, whether under professional supervision or self-administration.

The term “negligible amount” refers to a quantity, mass, or volume of a substance so minimal that it has no clinically, physiologically, or functionally significant effect for the intended purpose; for example, ≤125 mg of oral suspension or ≤25 mg of whitening agent being swallowed or washed away per treatment session.

The term “ingested” refers to entry of any portion of a material, intentionally or unintentionally, into the gastrointestinal tract of a patient via the oral route.

The term “slow-speed rotary handpiece” refers to any powered dental instrument, typically rotating at lower speeds (usually under 40,000 rpm), used in cleaning, preparation, or application procedures.

The term “rubber cup with abrasive paste” refers to rubber-based prophy cups, typically attached to a rotary handpiece, used with abrasive pastes to remove stains or deposits from teeth.

The term biofilm refers to any structured community of microorganisms-including bacteria, fungi, and matrix components-residing on or adhering to tooth, restoration, or oral surfaces, and often necessitating removal prior to whitening treatments.

The term “extrinsic staining present on the teeth” refers to any discoloration, staining, or surface color change on the outer surfaces of teeth resulting from exposure to chromogenic substances external to the tooth structure.

The term “intrinsic staining present on the teeth” refers to any internal discoloration originating from within the tooth's enamel or dentin, typically not amenable to removal by surface cleaning alone and requiring bleaching agents capable of penetrating into the tooth structure.

The term “toothpaste” refers to any dentifrice in paste, gel, or powder form, containing abrasives, detergents, or additives, used for cleaning teeth.

The term “ultrasonic scaler” refers to a powered dental instrument equipped with ultrasonic vibration to remove plaque, calculus, or stains from tooth surfaces.

The term “hand instruments” refers to manually operated dental tools used for debridement, cleaning, or preparation of teeth and oral tissues.

The term “flossing” refers to the process of cleaning interproximal surfaces of teeth using dental floss or analogs.

The term “plaque” refers to adherent soft deposits consisting primarily of microorganisms, matrix, and byproducts on tooth surfaces.

The term “light energy” refers to any electromagnetic radiation in the visible, ultraviolet, or near-infrared spectrum capable of activating tooth whitening agents, including but not limited to specified light sources herein.

The term “heat” refers to thermal energy applied to the teeth or dental varnish, by conduction, convection, or radiation, for the purpose of accelerating chemical reactions or material transformation.

The term “UV” refers to ultraviolet light in any range applicable for dental procedures (approximately 10-400 nm wavelength).

The term “Halogen” refers to halogen lamp light sources used to provide broad-spectrum or filtered light energy in dental procedures.

The term “LED” refers to light-emitting diode or diode arrays providing visible or near-ultraviolet light for activating tooth whitening or setting materials.

The term “plasma arc” refers to a dental light source based on plasma discharge, producing high-intensity visible or ultraviolet light suited for activating dental procedures.

The term “pulp of the teeth” refers to the living connective tissue core of a tooth, including nerves and blood vessels, contained within the dentin and enamel.

The term “applicator” refers to any tool, implement, or device used to deliver, spread, or position the dental composition on teeth, including brushes, swabs, syringes, or other means.

The term “kit” refers to a packaged assembly containing the oral liquid dental suspension, optionally one or more applicators, instructions, or additional treatment components.

The term “applicator brush” refers to a brush instrument, with bristles formed of natural or synthetic filaments, used to apply and distribute compositions onto teeth.

The term “unidose packages” refers to individually portioned, sealed containers designed to deliver a single measured dose or treatment of composition.

The term “tube” refers to a container, typically cylindrical and flexible, used for storing and dispensing viscous or flowable liquids or gels.

The term “syringe dispenser” refers to any cylindrical vessel with a movable plunger, optionally with a needle or tip, for dispensing measured amounts of liquid or gel with manual or mechanical force.

The term “pen applicator” refers to a device generally resembling a pen, containing a reservoir of product and a tip or brush for precise application to target areas.

The term “prescribing information” refers to written, printed, or electronic instructions containing usage directions, safety data, or regulatory compliance information supplied with the product.

The term “mouthpiece” refers to any intraoral device, tray, or form-fitting structure designed to engage the teeth and/or oral tissues for the administration or activation of dental treatments, optionally integrated with light or heat sources.

The term “autoclavable illuminate heat mouthpiece” refers to a mouthpiece device configured to withstand sterilization by autoclave and incorporating elements providing illumination and/or thermal energy to teeth.

The term “case” refers to any container, box, or enclosure suitable for storage or transport of one or more items in the kit, including applicators, mouthpieces, and associated equipment.

The term “power control with built-in timer” refers to a device or system that regulates power supply to treatment equipment and provides an integrated timer to control duration of use.

The term “portable charging base” refers to a standalone charging unit, typically rechargeable and transportable, for providing electrical power to devices in the kit.

The term “power adapter & USB cable” refers to electrical components, including adapters compatible with AC or DC outlets and associated USB cables, for providing or adapting electrical power to devices in the kit.

The term “user manual” refers to an instruction set, whether printed or digital, that describes product operation, safety, indications, and any other usage or regulatory information for the kit or its components.

### Dental Varnish System for Advanced Teeth Whitening

The present invention introduces a groundbreaking approach to the field of teeth whitening, focusing on a dental varnish system that offers significant advances in both technology and patient experience. The present invention encompasses novel topical compositions, versatile methods of use, and integrated kit solutions, each designed to address technical limitations and user challenges found in conventional whitening systems.

### Topical Composition

Central to the present invention is the formulation of an oral liquid dental suspension that dries rapidly in the oral cavity, forming a discrete and tenacious varnish on the tooth surface. The composition includes a tooth whitening agent, a suitable solvent, and at least one resin or urethane, creating a medium that ensures strong adhesion, controlled release, and extended contact time with tooth enamel. Whitening agents such as hydrogen peroxide, carbamide peroxide, or non-peroxide oxidizers like phthalimidoperoxycaproic acid (PAP) are incorporated at therapeutically effective concentrations and can be further stabilized with optional agents such as EDTA or cyclodextrin. The inventive suspension can include a variety of excipients and additives, such as humectants, desensitizers (e.g., potassium nitrate, nano-hydroxyapatite), remineralization agents, glazing agents, flavoring components, and visual indicators, all of which contribute to efficacy, user comfort, and safety.

The use of film-forming resins or urethane monomers distinguishes the composition by enabling it to rapidly set-even in the presence of saliva-yielding a varnish that firmly adheres to the teeth. This strong adhesion mitigates premature loss by oral fluids or accidental ingestion and ensures a sustained and precise delivery of active agents. The formulation supports the inclusion of encapsulated or matrix-bound whitening agents, controlled-release mechanisms, as well as tailored physical properties such as non-dripping, string-free, and clump-free application, resulting in a uniform, continuous film with optimized viscosity and film thickness. Moreover, the dental suspension can be formulated as vegan, cruelty-free, free of common allergens, and adapted for sensitive patient groups or professional and non-professional use, further extending its accessibility and appeal.

### Methods of Use

The present invention presents versatile methods for the application and utilization of the dental varnish in medical, dental, and cosmetic procedures. These methods emphasize simplicity and efficiency: the suspension is readily applied to the teeth—whether all, some, or a single tooth—via brushing, painting, or spraying. Upon application, the product dries in seconds, forming a varnish that retains its effectiveness and contact with the enamel typically for periods ranging from minutes to several hours. This extended contact time enables the whitening agent to penetrate and act on both extrinsic and intrinsic tooth stains, delivering noticeable and lasting results while minimizing common drawbacks such as sensitivity and gingival irritation.

The methods further enable selective or comprehensive tooth treatment, gag-free application (critical for tray-sensitive or orthodontic patients), and compatibility with both professional dental settings and at-home regimens. Notably, the varnish can be used in conjunction with external light or heat sources—including ultraviolet, halogen, LED, or plasma arc devices—to further accelerate or enhance the whitening process. The present invention accommodates the use of pre- and post-bleaching rinses or treatments containing remineralizing, desensitizing, or fluoridating agents, supporting holistic patient care and maximizing dental health. The composition's strong adhesion also provides a temporary protective barrier that allows continued consumption of staining beverages with reduced risk of post-treatment discoloration.

### Kits and Integrated Systems

The present invention is also embodied in comprehensive kits that combine the oral liquid dental suspension with a variety of delivery and accessory components. These kits may include single-use packaging such as tubes, syringe dispensers, pen applicators, or blister packs, ensuring precise dosage, hygiene, and convenience. Optional accessories include specialized applicators, autoclavable mouthpieces with built-in light or heat sources, power controllers with integrated timers, portable charging solutions, and digital shade guides. Kits can be further augmented with oral rinses for pre-treatment or post-treatment use, as well as user-friendly instructions and digital support tools for tracking and customizing treatments.

By providing modular solutions, the kits empower both dental professionals and consumers to achieve controlled, effective, and safe whitening results without the need for complex trays or in-office supervision. The inclusion of digital or smart accessories supports advances in patient engagement, progress tracking, and individualized care.

### Principal Advantages and Applications

The dental varnish technology outlined in this present invention offers a compelling set of advantages compared to traditional whitening methods. Foremost, it enables prolonged and targeted delivery of whitening agents with minimal risk of ingestion or premature loss, thus maximizing efficacy while reducing side effects. Its flexible formulation framework supports the integration of therapeutic, protective, and sensory-enhancing agents, making it broadly suitable for a wide spectrum of users and indications, including children, adolescents, and individuals with dental restorations or sensitivities.

The varnish's robust, moisture-activated adhesion and rapid setting reduces treatment time and streamlines both home and professional applications. The potential to pair with light or heat activation, remineralizing rinses, and digital tracking further expands its therapeutic and commercial potential. These features collectively position the invention as a significant advancement in teeth whitening technology, establishing a new standard for efficacy, convenience, and patient safety in both consumer and clinical markets

### Specific Ranges, Values, and Embodiments

The specific embodiments describing the ranges and values provided below are for illustration purposes only, and do not otherwise limit the scope of the disclosed subject matter, as defined by the claims.

### Whitening Agents

In specific embodiments, the tooth whitening agent includes phthalimidoperoxycaproic acid (PAP), hydrogen peroxide, carbamide peroxide, sodium perborate, sodium percarbonate, sodium persulfate, calcium peroxide, magnesium peroxide, urea peroxide, zinc peroxide, or any combination thereof.

In specific embodiments, the tooth whitening agent is present at 0.1-45 wt. % of the suspension.

In specific embodiments, the tooth whitening agent includes 1-25 wt. % PAP.

In specific embodiments, the tooth whitening agent includes 2-45 wt. % hydrogen peroxide.

In specific embodiments, the tooth whitening agent includes 10-44 wt. % carbamide peroxide.

In specific embodiments, the tooth whitening agent includes a non-peroxide oxidizing agent.

In specific embodiments, the tooth whitening agent is encapsulated for controlled release.

In specific embodiments, the oral liquid dental suspension is free of peroxide compounds.

In specific embodiments, the tooth whitening agent is stabilized by the presence of EDTA, cyclodextrin, or other chelators.

In specific embodiments, the oral liquid dental suspension includes encapsulated or matrix-bound whitening agent for sustained, continuous, delayed, or pulsatile release over an extended period (e.g., several hours to days).

### Solvents

In specific embodiments, the solvent includes ethanol, glycerin, propylene glycol, water, or any combination thereof.

In specific embodiments, ethanol is present at 5-40 wt. %.

In specific embodiments, the oral liquid dental suspension is substantially anhydrous.

In specific embodiments, the solvent includes propylene glycol in a concentration of 2-12 wt. %.

In specific embodiments, the solvent includes a polyol, such as xylitol or sorbitol, in addition to a lower alkanol.

### Resins and Polymeric Film-Formers

In specific embodiments, the resin includes colophony (rosin), hydrogenated rosin, polyvinyl acetate, mastic, methyl esters of hydrogenated rosin, synthetic resins (CAS No. 68990-02-3), pentaerythritol glycerol ester of colophony, or shellac (E904).

In specific embodiments, the resin is present at 10-80 wt. %.

In specific embodiments, shellac is present at 10-40 wt. %.

In specific embodiments, the resin includes 30-50 wt. % polyvinyl acetate and 10-30 wt. % hydrogenated rosin.

In specific embodiments, the resin is a synthetic methacrylate-based resin.

In specific embodiments, the resin is a synthetic resin selected to provide a film thickness less than 100 μm after drying.

In specific embodiments, the urethane includes urethane dimethacrylate at 1-25 wt. %.

In specific embodiments, the base includes both natural resin and synthetic resin at a combined concentration of 30-60 wt. %.

### Glazing Agents

In specific embodiments, the oral liquid dental suspension further includes a glazing agent.

In specific embodiments, the glazing agent including at least one of white wax (E901), beeswax, carnauba wax, candelilla wax, microcrystalline wax, and shellac, alone or in combination.

In specific embodiments, the glazing agent is present at 0.1-10 wt. %.

### Additives for Patient Experience and Efficacy

In specific embodiments, the oral liquid dental suspension includes a sweetener, such as xylitol, sucralose, sorbitol, sodium saccharin, or combinations thereof.

In specific embodiments, the oral liquid dental suspension includes a flavoring agent, such as peppermint oil, spearmint, menthol, thymol, eucalyptol, fruit flavor, caramel, or bubblegum flavor.

In specific embodiments, the flavoring agent is present at 0.01-3 wt. %.

In specific embodiments, the suspension includes a colorant, such as titanium dioxide, mica, or plant-derived pigments.

### Desensitizing and Remineralizing Agents

In specific embodiments, the oral liquid dental suspension includes a teeth desensitizer including at least one of potassium nitrate, potassium citrate, tricalcium phosphate, tetrapotassium pyrophosphate, and hydroxyapatite.

In specific embodiments, the oral liquid dental suspension includes nano-hydroxyapatite particles of less than 100 nm average particle size.

In specific embodiments, the oral liquid dental suspension includes a remineralization agent at 0.05-10 wt. %.

### Controlled Release and Stability Enhancements

In specific embodiments, the bleaching agent is encapsulated in a polymeric matrix, microspheres, nanofibers, or in micro/nanocapsules for controlled release.

In specific embodiments, the oral liquid dental suspension has a pH of less than 5, or preferably between 3 and 4.5, stabilized by citric acid or phosphoric acid.

In specific embodiments, EDTA is present at 0.01-2.5 wt. % as a sequestering or stabilizing agent.

In specific embodiments, the bleaching agent is protected in a two-phase composition, wherein the varnish and bleaching agent are mixed at the time of use.

### Application and Use Methods

In specific embodiments, the method includes applying the oral liquid dental suspension to the teeth with a brush, swab, sprayer, pen, or aerosol.

In specific embodiments, the method includes delivering the oral liquid dental suspension to the teeth as a liquid, spray, foam, or aerosol that adheres and forms a varnish-like layer on teeth.

In specific embodiments, the method includes delivering the oral liquid dental suspension to the teeth as a liquid with a mouthpiece with automated dosing.

In specific embodiments, the method includes applying the oral liquid dental suspension to cleaned, dried teeth, optionally after surface preparation by rotary cup, hand instrumentation, or air abrasion.

In specific embodiments, the method further includes applying an adhesive layer to the teeth prior to applying the oral liquid dental suspension, for enhanced retention.

In specific embodiments, the varnish is dries ≤120 seconds in the presence of saliva.

In specific embodiments, the varnish is dries ≤90 seconds in the presence of saliva.

In specific embodiments, the varnish is dries ≤75 seconds in the presence of saliva.

In specific embodiments, the varnish is dries ≤60 seconds in the presence of saliva.

In specific embodiments, the varnish is dries ≤45 seconds in the presence of saliva.

In specific embodiments, the varnish is dries ≤30 seconds in the presence of saliva.

In specific embodiments, the varnish is dries ≤25 seconds in the presence of saliva.

In specific embodiments, the varnish is dries ≤20 seconds in the presence of saliva.

In specific embodiments, the varnish is dries ≤15 seconds in the presence of saliva.

In specific embodiments, the varnish is dries ≤10 seconds in the presence of saliva.

In specific embodiments, the varnish is dries ≤5 seconds in the presence of saliva.

In specific embodiments, the varnish dries quickly (e.g., within 5, 15, or 30 seconds) in the presence of saliva.

In specific embodiments, the varnish adheres and remains effective for 0.1-12 hours after application.

In specific embodiments, the method includes applying the oral liquid dental suspension without the use of trays or bite guards, for gag-free application.

In specific embodiments, the method includes exposing the varnish to light energy including at least one of UV, halogen, LED, and plasma arc after application, for 0.05-0.5 hours.

In specific embodiments, the method includes multiple, sequential applications to selected teeth for targeted whitening.

In specific embodiments, the method further includes a post-bleaching step of applying a remineralizing or desensitizing oral rinse.

In specific embodiments, the method includes applying the oral liquid dental suspension to dental prosthetics, restorations, implants, crowns, or veneers.

In specific embodiments, with the method described herein, the varnish effectively providing a temporary barrier against stains from beverages such as coffee, tea, wine, or soda after whitening.

In specific embodiments, the method is accompanied by a negligible amount of oral liquid dental suspension being swallowed by the patient, such that 50-125 mg oral liquid dental suspension is swallowed by the patient, in a session.

In specific embodiments, the method is accompanied by a negligible amount of oral liquid dental suspension being swallowed by the patient, such that ≤125 mg oral liquid dental suspension is swallowed by the patient, in a session.

In specific embodiments, the method is accompanied by a negligible amount of oral liquid dental suspension being swallowed by the patient, such that ≤100 mg oral liquid dental suspension is swallowed by the patient, in a session.

In specific embodiments, the method is accompanied by a negligible amount of oral liquid dental suspension being swallowed by the patient, such that ≤75 mg oral liquid dental suspension is swallowed by the patient, in a session.

In specific embodiments, the method is accompanied by a negligible amount of oral liquid dental suspension being swallowed by the patient, such that ≤50 mg oral liquid dental suspension is swallowed by the patient, in a session.

In specific embodiments, the method is accompanied by a negligible amount of oral liquid dental suspension being swallowed by the patient, such that ≤25 mg oral liquid dental suspension is swallowed by the patient, in a session.

In specific embodiments, the method is accompanied by a negligible amount of oral liquid dental suspension being swallowed by the patient, such that ≤10 mg oral liquid dental suspension is swallowed by the patient, in a session.

In specific embodiments, the method is accompanied by a negligible amount of tooth whitening agent being swallowed by the patient, such that 5-25 mg tooth whitening agent is swallowed by the patient, in a session.

In specific embodiments, the method is accompanied by a negligible amount of tooth whitening agent being swallowed by the patient, such that ≤25 mg tooth whitening agent is swallowed by the patient, in a session.

In specific embodiments, the method is accompanied by a negligible amount of tooth whitening agent being swallowed by the patient, such that ≤20 mg tooth whitening agent is swallowed by the patient, in a session.

In specific embodiments, the method is accompanied by a negligible amount of tooth whitening agent being swallowed by the patient, such that ≤15 mg tooth whitening agent is swallowed by the patient, in a session.

In specific embodiments, the method is accompanied by a negligible amount of tooth whitening agent being swallowed by the patient, such that ≤10 mg tooth whitening agent is swallowed by the patient, in a session.

In specific embodiments, the method is accompanied by a negligible amount of tooth whitening agent being swallowed by the patient, such that ≤5 mg tooth whitening agent is swallowed by the patient, in a session.

In specific embodiments, the method is accompanied by a negligible amount of tooth whitening agent being swallowed by the patient, such that ≤2.5 mg tooth whitening agent is swallowed by the patient, in a session.

In specific embodiments, the method is accompanied by a negligible amount of tooth whitening agent being washed away by saliva of the patient, such that 5-25 mg tooth whitening agent is s washed away by saliva of the patient, in a session.

In specific embodiments, the method is accompanied by a negligible amount of tooth whitening agent being washed away by saliva of the patient, such that ≤25 mg tooth whitening agent is washed away by saliva of the patient, in a session.

In specific embodiments, the method is accompanied by a negligible amount of tooth whitening agent being washed away by saliva of the patient, such that ≤20 mg tooth whitening agent is washed away by saliva of the patient, in a session.

In specific embodiments, the method is accompanied by a negligible amount of tooth whitening agent being washed away by saliva of the patient, such that ≤15 mg tooth whitening agent is washed away by saliva of the patient, in a session.

In specific embodiments, the method is accompanied by a negligible amount of tooth whitening agent being washed away by saliva of the patient, such that ≤10 mg tooth whitening agent is washed away by saliva of the patient, in a session.

In specific embodiments, the method is accompanied by a negligible amount of tooth whitening agent being washed away by saliva of the patient, such that ≤5 mg tooth whitening agent is washed away by saliva of the patient, in a session.

In specific embodiments, the method is accompanied by a negligible amount of tooth whitening agent being washed away by saliva of the patient, such that ≤2.5 mg tooth whitening agent is washed away by saliva of the patient, in a session.

### Packaging and Delivery Kits

In specific embodiments, the oral liquid dental suspension is provided in unidose packages, tubes, syringe dispensers, or pen applicators.

In specific embodiments, the kit optionally includes an autoclavable light- and/or heat-equipped mouthpiece, power controller with timer, charging base, and instructions for use.

In specific embodiments, the kit includes a separate pre-treatment desensitizing or remineralizing solution, a post-bleaching fluoridation rinse, and a shade guide.

In specific embodiments, the whitening kit contains a device for measuring color change or tooth shade before and after treatment.

In specific embodiments, the whitening kit contains digital shade guides, wireless sensors, smartphone apps, and/or Bluetooth-enabled devices for user feedback, timer control, or treatment customization.

### Specialized and Niche Embodiments

In specific embodiments, the oral liquid dental suspension is suitable for use by adolescents under 18 years of age.

In specific embodiments, the oral liquid dental suspension is formulated to produce minimal to no tooth sensitivity when used as directed.

In specific embodiments, the oral liquid dental suspension is free of animal-derived components or allergens (vegan or hypoallergenic formulation).

In specific embodiments, the oral liquid dental suspension is designed to be undetectable (clear or nearly clear) after drying on the teeth.

In specific embodiments, the oral liquid dental suspension is provided as an over-the-counter product, or as a prescription-only composition.

### Further Specific Formulations and Ranges

In specific embodiments, the suspension includes at least one resin at 10-30 wt. % and at least one glazing agent at 1-10 wt. %.

In specific embodiments, the whitening agent includes at least two bleaching agents in combination, e.g., hydrogen peroxide and PAP.

In specific embodiments, the resin includes a synthetic resin (e.g., polyvinyl acetate, methacrylate polymer) at 30-60 wt. %.

In specific embodiments, the oral liquid dental suspension is free of colorants and flavoring agents (“unflavored” embodiment), or is free of sweeteners (“unsweetened” embodiment).

In specific embodiments, the suspension includes 0.1-5 wt. % titanium dioxide as a catalyst or pigment.

In specific embodiments, the oral liquid dental suspension is formulated such that a visually perceptible color or luminescence is imparted to the treated tooth surface, or that the varnish is visible under UV light to aid application.

In specific embodiments, the oral liquid dental suspension includes a pH-buffering system enabling sustained release of active whitening agent at a pH between 3 and 7.

In specific embodiments, the varnish is designed to rapidly self-heal or redistribute if disturbed by salivary flow.

In specific embodiments, the oral liquid dental suspension is formulated with viscosity-modifying agents (e.g., ethyl cellulose) at 0.05-3 wt. % such that the viscosity is 200-800 centipoise.

### Advanced Ingredients and Additives

In specific embodiments, the oral liquid dental suspension includes bioactive natural compounds such as quercetin, theobromine, and tt-farnesol for anti-biofilm and remineralization activity.

In specific embodiments, the oral liquid dental suspension includes a chitosan-propolis blend for sustained antimicrobial and biofilm-disrupting effects.

In specific embodiments, the oral liquid dental suspension includes stabilizers such as butylated hydroxyanisole (BHA), butylated hydroxytoluene (BHT), tocopherols (Vitamin E), or rosemary extract to enhance bleaching agent stability.

In specific embodiments, the oral liquid dental suspension includes organo-phosphoric acid adhesion promoters with alkyl groups of 3-30 carbon atoms.

In specific embodiments, the oral liquid dental suspension further includes one or more enzymatic agents (e.g., papain, bromelain, or peroxidase) as adjuncts to the whitening agent for stain removal and/or oral health benefits.

In specific embodiments, the oral liquid dental suspension further includes one or more photoactive compounds (e.g., methylene blue, curcumin) that, when activated by visible or infrared light, enhance whitening and/or antimicrobial action.

In specific embodiments, the oral liquid dental suspension further includes one or more antimicrobial agents (e.g., silver nanoparticles, copper compounds, essential oils) or probiotics, for oral microbiome balance and/or improved oral health outcomes with whitening.

### Whitening and Remineralization Features

In specific embodiments, the varnish provides both tooth whitening and remineralizing effects via co-inclusion of biomineralizing peptides or nano-hydroxyapatite.

In specific embodiments, the oral liquid dental suspension enables high fluoride release (>14,000 μg/g in 2 hours) to maximize remineralization in a short wear time.

In specific embodiments, the oral liquid dental suspension has a dual-phase or multi-phase structure, with separated bleaching and remineralization agents mixed only at the time of use.

### Physical and Sensory Properties

In specific embodiments, the oral liquid dental suspension is substantially colorless and transparent upon drying for maximum patient acceptance.

In specific embodiments, the oral liquid dental suspension has a high-shear viscosity between 1,000-8,000 cP, enabling excellent retention and uniform film formation.

In specific embodiments, the varnish provides sustained release of active components (e.g., fluoride, propolis, bioactives) for at least 4-6 hours.

In specific embodiments, the oral liquid dental suspension has a pH-modulated formula, maintaining a nearly neutral pH for bleaching agent stability.

### Application and Delivery Enhancements

In specific embodiments, the oral liquid dental suspension can be applied without prior drying of teeth, setting within seconds even on moist enamel for ease-of-use in clinical or home settings.

In specific embodiments, the oral liquid dental suspension utilizes an adhesion-promoting agent (such as that in Enamelast™) for enhanced retention to teeth and soft tissue.

In specific embodiments, the oral liquid dental suspension is supplied in a dual-syringe or multi-compartment applicator to facilitate on-demand mixing of reactive ingredients.

### Special Functional Features

In specific embodiments, the oral liquid dental suspension is formulated for significantly reduced tooth sensitivity during and after whitening, via addition of potassium nitrate or citrate.

In specific embodiments, the oral liquid dental suspension contains visual tracers or color-change indicators for guidance during application but becomes clear and undetectable once set.

In specific embodiments, the oral liquid dental suspension forms a self-sealing film to protect against subsequent staining from coffee, tea, wine, or tobacco, post-treatment.

### Alternative Whitening and Cosmetic Uses

In specific embodiments, the oral liquid dental suspension is formulated for use as a finishing layer in dental bonding or veneer applications, to enhance brightness and seal against stains.

In specific embodiments, the formulation includes both whitening and desensitizing toothpaste/gel components for a multi-step care approach.

### Novel Packaging and Stability

In specific embodiments, the oral liquid dental suspension is packaged in air-tight, opaque, single-use blister packs for shelf life and dose control.

In specific embodiments, the oral liquid dental suspension includes antioxidants (e.g., propyl gallate or TBHQ) as shelf-life extenders for peroxide or non-peroxide actives.

In specific embodiments, the oral liquid dental suspension includes additional penetration enhancers or film-formers (e.g., polyvinylpyrrolidone, polysaccharides, or polyacrylates) for uniform coverage.

### Patient-Specific and Specialty Formulations

In specific embodiments, the oral liquid dental suspension is tailored for patients with chronic dry mouth (xerostomia) to provide additional lubrication and comfort.

In specific embodiments, the oral liquid dental suspension is formulated to be free of common allergens and is vegan and cruelty-free.

In specific embodiments, the oral liquid dental suspension is optimized for pediatric or geriatric use with lower peroxide levels, flavor customization, and easier handling.

### Antioxidant Stabilizers

In specific embodiments, the oral liquid dental suspension further includes one or more antioxidant stabilizers including at least one of butylated hydroxyanisole (BHA), butylated hydroxytoluene (BHT), tocopherols (e.g., Vitamin E), rosemary extract, propyl gallate, and tert-butylhydroquinone (TBHQ), for enhancing shelf life and peroxide stability.

### High Water Content and Humectant Balance

In specific embodiments, the oral liquid dental suspension includes at least 20 wt. % water by weight, or includes a high-humidity humectant system such as glycerin, propylene glycol, or a mixture thereof to maintain tooth moisture and reduce sensitivity during whitening.

In specific embodiments, the total humectant content (including water, glycerin, propylene glycol, sorbitol, xylitol, or combinations thereof) is present at 15-70 wt. % of the suspension.

### Polymeric Thickeners and Film-Formers

In specific embodiments, the oral liquid dental suspension includes a polymeric thickener and/or film-forming agent including at least one of polyvinylpyrrolidone (PVP), polyethylene oxide, polyacrylic acid, sodium carboxymethylcellulose, hydroxyethylcellulose, methylcellulose, and polysaccharides, in addition to or in place of the resin.

In specific embodiments, the polymeric thickener is present at 0.1-20 wt. % to control viscosity, uniformity, and film retention on teeth.

### Hypoallergenic, Specialty, and Lifestyle Formulations

In specific embodiments, the oral liquid dental suspension is gluten-free, sodium lauryl sulfate (SLS)-free, dye-free, and free of animal-derived ingredients, making the formulation vegan, kosher, Halal, or suitable for patients with allergies or dietary restrictions.

In specific embodiments, the oral liquid dental suspension is formulated as cruelty-free, or with only synthetic or plant-derived excipients.

### String-Free, Clump-Free, and Controlled Drip Application

In specific embodiments, the oral liquid dental suspension is engineered for string-free, clump-free, and non-drip performance during application via viscosity optimization or rheological modifiers, allowing for precise, uniform, and clean application onto tooth surfaces.

### Color-Change Visual Application Indicators

In specific embodiments, the oral liquid dental suspension includes a color-change or visual tracer additive (including dyes, pigments, or UV-fluorescent tracers) that is visible during application for enhanced placement accuracy and which becomes transparent, undetectable, or visually non-significant once the varnish is dry and set.

### Moisture-Activated, Low-Temperature, and Dual-Cure Setting

In specific embodiments, the oral liquid dental suspension is formulated to set or cure upon exposure to saliva, oral moisture, or ambient temperature in the oral cavity, eliminating any need for external heating or pre-drying of teeth.

In specific embodiments, the oral liquid dental suspension utilizes a dual-cure or multi-cure system that may be activated by light, moisture, or a combination thereof to accelerate setting and optimize contact time.

### Single-Dose, Pen, and Blister-Pack Packaging

In specific embodiments, the oral liquid dental suspension is packaged and offered in single-use, unit-dose containers, air-tight blister packs, or self-dispensing applicator pens for optimal infection control, dose control, and convenience.

### Multi-Phase and PH-Balanced Release Systems

In specific embodiments, the oral liquid dental suspension includes a dual-phase or multi-phase system, wherein the whitening agent and remineralizing or desensitizing agents are separated until the time of use and combined prior to or during application.

In specific embodiments, the oral liquid dental suspension contains a pH-buffering system enabling sustained release of the active whitening agent at a controlled pH between 3 and 7, with the pH being instantaneously or gradually modifiable upon setting.

### Fast-Setting and Early Activity Resumption

In specific embodiments, the oral liquid dental suspension is formulated to set and form a protective, retentive varnish within 1-5 minutes of application, permitting the patient to resume eating, drinking, or routine oral activity shortly after treatment while maintaining whitening efficacy.

### Whitening Agent Content

In specific embodiments, the tooth whitening agent is present at 0.1-45 wt. % of the suspension.

In specific embodiments, the tooth whitening agent is present at 5-20 wt. % of the suspension.

In specific embodiments, the PAP (phthalimidoperoxycaproic acid) is present at 1-25 wt. % of the suspension.

In specific embodiments, the PAP (phthalimidoperoxycaproic acid) is present at 5-12 wt. % of the suspension.

In specific embodiments, the hydrogen peroxide is present at 2-45 wt. % of the suspension.

In specific embodiments, the hydrogen peroxide is present at 25-40 wt. % of the suspension.

In specific embodiments, the hydrogen peroxide is present at 3-10 wt. %, or 15-35 wt. % of the suspension.

In specific embodiments, the carbamide peroxide is present at 10-44 wt. % of the suspension.

In specific embodiments, the carbamide peroxide is present at 10-16 wt. % of the suspension.

In specific embodiments, the carbamide peroxide is present at 2-6 wt. % of the suspension.

In specific embodiments, the carbamide peroxide is present at 10-16 wt. %, or up to 35 wt. % of the suspension.

### Solvent and Humectant Content

In specific embodiments, the solvent is present at 2-40 wt. % of the suspension.

In specific embodiments, the solvent is present at 10-30 wt. % of the suspension.

In specific embodiments, the ethanol is present at 5-40 wt. % of the suspension.

In specific embodiments, the ethanol is present at 10-30 wt. %, 10-15 wt. %, 1-15 wt. %, 20-30 wt. %, or 20-40 wt. % of the suspension.

In specific embodiments, the ethanol is present at 10-20 wt. % of the suspension.

In specific embodiments, the propylene glycol is present at 2-12 wt. % of the suspension.

In specific embodiments, the propylene glycol is present at 2-6 wt. % of the suspension.

In specific embodiments, the water is present at ≥20 wt. % of the suspension.

In specific embodiments, the water is present at 30-35 wt. % of the suspension.

In specific embodiments, the humectant (including water, glycerin, propylene glycol, sorbitol, xylitol, or combinations thereof) content is 15-70 wt. % of the suspension.

In specific embodiments, the humectant (including water, glycerin, propylene glycol, sorbitol, xylitol, or combinations thereof) content is 20-40 wt. % of the suspension.

### Resin and Polymeric Film-Formers Content

In specific embodiments, the resin is present at 10-80 wt. % of the suspension.

In specific embodiments, the resin is present at 20-35 wt. % of the suspension.

In specific embodiments, the hydrogenated rosin is present at 10-30 wt. % of the suspension.

In specific embodiments, the hydrogenated rosin is present at 10-25 wt. % of the suspension.

In specific embodiments, the polyvinyl acetate is present at 15-50 wt. % of the suspension.

In specific embodiments, the polyvinyl acetate is present at 15-30 wt. % of the suspension.

In specific embodiments, the colophonium is present at 30-60 wt. % of the suspension.

In specific embodiments, the colophonium is present at 10-20 wt. % of the suspension.

In specific embodiments, the pentaerythritol glycerol ester of colophony resin is present at 30-75 wt. % of the suspension.

In specific embodiments, the rosin is present at 10-30 wt. % of the suspension.

In specific embodiments, the shellac is present at 10-40 wt. % of the suspension.

In specific embodiments, the shellac is present at 10-30 wt. % of the suspension.

In specific embodiments, the synthetic resin (e.g., polyvinyl acetate, methacrylate polymer) is present at 30-60 wt. % of the suspension.

In specific embodiments, the synthetic resin (e.g., polyvinyl acetate, methacrylate polymer) is present at 20-40 wt. % of the suspension.

In specific embodiments, the urethane (urethane dimethacrylate) is present at 1-25 wt. % of the suspension.

In specific embodiments, the urethane (urethane dimethacrylate) is present at 1-10 wt. % of the suspension.

In specific embodiments, the base includes both natural and synthetic resin at a combined amount of 20-60 wt. % of the suspension.

In specific embodiments, the base includes both natural and synthetic resin at a combined amount of 20-40 wt. % of the suspension.

In specific embodiments, the polymeric thickener is present at 0.1-20 wt. % of the suspension.

In specific embodiments, the polymeric thickener is present at 0.5-5 wt. % of the suspension.

### Glazing Agent Content

In specific embodiments, the glazing agent is present at 0.1-10 wt. % of the suspension.

In specific embodiments, the glazing agent is present at 0.5-2 wt. % of the suspension.

### Flavor Content

In specific embodiments, the flavoring agent is present at 0.01-3 wt. % of the suspension.

In specific embodiments, the flavoring agent is present at 0.1-1 wt. % of the suspension.

### Shellac Content

In specific embodiments, the shellac is present at 25-35 wt. % of the suspension, to achieve desirable viscosity.

In specific embodiments, the shellac is present at 25-30 wt. % of the suspension, to achieve desirable viscosity.

Titanium Dioxide as a Catalyst and/or Pigment

In specific embodiments, the titanium dioxide is present at 0.1-5 wt. % of the suspension.

In specific embodiments, the titanium dioxide is present at 0.1-2.5 wt. % of the suspension.

In specific embodiments, the titanium dioxide is present at 0.1-1 wt. % of the suspension.

### Desensitizing and Remineralizing Agents

In specific embodiments, the remineralization agent is present at 0.05-10 wt. % of the suspension.

In specific embodiments, the remineralization agent is present at 1-10 wt. % of the suspension.

In specific embodiments, the remineralization agent is present at 1-5 wt. % of the suspension.

In specific embodiments, the desensitizing agent is present at 0.1-10 wt. % of the suspension.

In specific embodiments, the desensitizing agent is present at 1-5 wt. % of the suspension.

### Sequestering and Complexing Agent

In specific embodiments, the EDTA (sequestering agent) is present at 0.01-2.5 wt. % of the suspension.

In specific embodiments, the EDTA (sequestering agent) is present at 0.05-0.5 wt. % of the suspension.

In specific embodiments, the cyclodextrin (complexing agent) is present at 0.01-2.5 wt. % of the suspension.

In specific embodiments, the cyclodextrin (complexing agent) is present at 0.1-1 wt. % of the suspension.

### Polymers and Viscosity Agents

In specific embodiments, the ethyl cellulose is present at 0.05-3 wt. % of the suspension.

In specific embodiments, the ethyl cellulose is present at 0.1-1 wt. % of the suspension.

### pH and Buffer Systems

In specific embodiments, the suspension has a pH of ≤7.

In specific embodiments, the suspension has a pH of ≤6.5.

In specific embodiments, the suspension has a pH of ≤6.

In specific embodiments, the suspension has a pH of ≤5.5.

In specific embodiments, the suspension has a pH of ≤5.

In specific embodiments, the suspension has a pH of ≤4.5.

In specific embodiments, the suspension has a pH of ≤4.

In specific embodiments, the suspension has a pH of 3-7.

In specific embodiments, the suspension has a pH of 4-6.5.

In specific embodiments, the suspension has a pH of 3-4.5.

### Bleach Activator

In specific embodiments, the bleach activator (TAED, NOBS, etc.) is present up to 0.1 wt. % of the liquid solution.

In specific embodiments, the bleach activator (TAED, NOBS, etc.) is present at 0.01-0.1 wt. % of the liquid solution.

In specific embodiments, the bleach activator (TAED, NOBS, etc.) is present at 0.01-0.05 wt. % of the liquid solution.

### Film Thickness

In specific embodiments, the film thickness is less than 100 μm after drying.

In specific embodiments, the film thickness is less than 5-50 μm after drying.

### Nano-Hydroxyapatite Particles

In specific embodiments, the nano-hydroxyapatite particles have an average particle size less than 100 nm.

In specific embodiments, the nano-hydroxyapatite particles have an average particle size of 20-80 nm.

### Viscosity

In specific embodiments, the suspension has a viscosity of 200-800 centipoise.

In specific embodiments, the suspension has a viscosity of 300-600 centipoise.

### High-Shear Viscosity

In specific embodiments, the suspension has a high-shear viscosity of 1,000-8,000 cP.

In specific embodiments, the suspension has a high-shear viscosity of 2,000-5,000 cP.

### Application Contact Times

In specific embodiments, the varnish is in contact with the surface of the teeth for 0.1-12 hours.

In specific embodiments, the varnish forms a coating for teeth whitening (bleaching) and stays essentially intact for 0.1-12 hours.

In specific embodiments, the varnish forms a coating for teeth whitening (bleaching) and stays essentially intact for 0.25-2 hours.

In specific embodiments, the varnish forms a coating for teeth whitening (bleaching) and stays essentially intact for 0.25-1 hour.

In specific embodiments, the varnish forms a coating for teeth whitening (bleaching) and stays essentially intact for 0.5-2 hours.

### Drying Time

In specific embodiments, the varnish sets (dries) to form a film ≤90 seconds.

In specific embodiments, the varnish sets (dries) to form a film ≤75 seconds.

In specific embodiments, the varnish sets (dries) to form a film ≤60 seconds.

In specific embodiments, the varnish sets (dries) to form a film ≤45 seconds.

In specific embodiments, the varnish sets (dries) to form a film ≤30 seconds.

In specific embodiments, the varnish sets (dries) to form a film ≤20 seconds.

In specific embodiments, the varnish sets (dries) to form a film ≤10 seconds.

In specific embodiments, the varnish sets (dries) to form a film ≤5 seconds.

Exposure to Light Energy (after Application)

In specific embodiments, the varnish is exposed to light energy for 0.05-0.5 hours.

In specific embodiments, the varnish is exposed to light energy for 0.05-0.25 hours.

In specific embodiments, the varnish is exposed to light energy for 0.05-0.20 hours.

In specific embodiments, the varnish is exposed to light energy for 0.05-0.15 hours.

In specific embodiments, the varnish is exposed to light energy for 0.05-0.10 hours.

### Post-Treatment Mouth Rinsing

In specific embodiments, 0.1-1.5 hours after application the mouth is rinsed.

In specific embodiments, 0.25-0.5 hours after application the mouth is rinsed.

### Treatment (Administration) Frequency

In specific embodiments, the suspension is applied once a year.

In specific embodiments, the suspension is applied up to 25 times a year.

In specific embodiments, the suspension is applied up to 12 times a year.

In specific embodiments, the suspension is applied once every six months.

In specific embodiments, the suspension is applied once a month.

In specific embodiments, the suspension is applied once a day.

In specific embodiments, the suspension is applied once a day for 2-15 consecutive days.

In specific embodiments, the suspension is applied at least twice daily.

In specific embodiments, the suspension is applied at least twice daily for 2-15 consecutive days.

In specific embodiments, the suspension is applied once daily for up to 14 days per course; touch-ups monthly.

In specific embodiments, the suspension is applied 1-3 sessions per year.

### ENUMERATED EMBODIMENTS

Specific enumerated embodiments <1> to <175> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### Composition (Oral Liquid Dental Suspension)

<Embodiment 1> An oral liquid dental suspension including (a) a tooth whitening agent, (b) a solvent, and (c) at least one of a resin and a urethane.

<Embodiment 2> An oral liquid dental suspension including (a) a tooth whitening agent including at least one of peroxide-based and non-peroxide-based oxidizers, (b) a solvent including at least one of ethanol, water, glycerin, and propylene glycol, and (c) a resin including at least one of a synthetic and natural resin.

<Embodiment 3> An oral liquid dental suspension including a tooth whitening agent, a solvent, and a film-forming resin, wherein the composition is formulated to set to a varnish in the presence of saliva.

<Embodiment 4> An oral liquid dental suspension including (a) a tooth whitening agent, (b) a solvent system containing at least one hydrophilic and one hydrophobic solvent, and (c) a resin base providing rapid film formation on tooth surfaces.

<Embodiment 5> An oral liquid dental suspension including a tooth whitening agent, a solvent, and a polymeric resin, wherein upon application to human teeth the suspension, in the presence of saliva, dries within 90 seconds to form a continuous, retentive varnish layer.

<Embodiment 6> An oral liquid dental suspension including (a) hydrogen peroxide or carbamide peroxide as a tooth whitening agent, (b) ethanol as a solvent, and (c) a synthetic resin including at least one of polyvinyl acetate and methacrylate polymer.

<Embodiment 7> An oral liquid dental suspension including (a) phthalimidoperoxycaproic acid (PAP) as a tooth whitening agent, (b) ethanol and propylene glycol as solvents, (c) shellac or hydrogenated rosin as film-forming resins.

<Embodiment 8> An oral liquid dental suspension including a tooth whitening agent at 0.1-45 wt. %, solvent at 2-40 wt. %, and resin at 10-80 wt. %.

<Embodiment 9> An oral liquid dental suspension including a tooth whitening agent encapsulated within a polymeric matrix providing controlled release over 0.1-12 hours when applied to tooth surfaces.

<Embodiment 10> An oral liquid dental suspension including a non-peroxide oxidizing agent as a tooth whitening agent, a solvent, and a resin base, wherein the suspension is free of animal-derived ingredients and sodium lauryl sulfate.

<Embodiment 11> An oral liquid dental suspension including (a) a combination of hydrogen peroxide and phthalimidoperoxycaproic acid as tooth whitening agents, (b) polyvinyl acetate and shellac as resins, and (c) ethanol and propylene glycol as solvents.

<Embodiment 12> An oral liquid dental suspension including (a) a tooth whitening agent, (b) a solvent, (c) a resin, and (d) a remineralization agent including at least one of nano-hydroxyapatite and tricalcium phosphate.

<Embodiment 13> An oral liquid dental suspension including (a) a tooth whitening agent, (b) a solvent, (c) a resin, and (d) a glazing agent including at least one of white wax, carnauba wax, and microcrystalline wax.

<Embodiment 14> An oral liquid dental suspension including (a) a tooth whitening agent, (b) a solvent, (c) a resin, and (d) a sweetener including at least one of xylitol and sucralose.

<Embodiment 15> An oral liquid dental suspension including (a) a tooth whitening agent stabilized by ethylenediaminetetraacetic acid (EDTA) at 0.01-2.5 wt. %, (b) a solvent, (c) a resin, and (d) a flavoring agent.

<Embodiment 16> An oral liquid dental suspension including (a) a tooth whitening agent, (b) a solvent, (c) a resin, and (d) a color-change indicator for visual guidance during application, which fades or disappears when the varnish is set.

<Embodiment 17> An oral liquid dental suspension including (a) hydrogen peroxide at 2-45 wt. % as a tooth whitening agent, (b) ethanol as a solvent at 5-40 wt. %, and (c) shellac at 25-35 wt. % as a resin.

<Embodiment 18> An oral liquid dental suspension including (a) carbamide peroxide at 10-44 wt. % as a tooth whitening agent, (b) glycerin and propylene glycol as solvents, and (c) hydrogenated rosin as a resin.

<Embodiment 19> An oral liquid dental suspension including (a) phthalimidoperoxycaproic acid (PAP) at 1-12 wt. % and hydrogen peroxide at 3-10 wt. %, (b) a synthetic resin base including shellac at 25-35 wt. % and hydrogenated rosin at 10-25 wt. %, (c) at least one glazing agent at 0.1-2 wt. %, and (d) nano-hydroxyapatite at 0.05-5 wt. %.

<Embodiment 20> An oral liquid dental suspension including (a) a tooth whitening agent, (b) a solvent, (c) a resin, (d) a desensitizing agent including at least one of potassium nitrate and potassium citrate, at 0.1-5 wt. %, and (e) a pH adjusting agent maintaining a pH between 3.5 and 4.5.

<Embodiment 21> An oral liquid dental suspension including (a) hydrogen peroxide as a tooth whitening agent at 2-45 wt. %, (b) ethanol as a solvent at 10-30 wt. %, (c) polyvinyl acetate at 15-50 wt. % as a resin, and (d) titanium dioxide at 0.1-5 wt. % as a catalyst or pigment.

<Embodiment 22> An oral liquid dental suspension including (a) a whitening agent encapsulated in microspheres, (b) a solvent, (c) a resin including at least one of shellac and methacrylate polymer, and (d) a viscosity modifier including ethyl cellulose at 0.05-3 wt. %.

<Embodiment 23> An oral liquid dental suspension including (a) PAP as a tooth whitening agent, (b) nano-hydroxyapatite particles under 100 nm as a remineralization agent, and (c) potassium citrate as a desensitizer.

<Embodiment 24> An oral liquid dental suspension including (a) hydrogen peroxide, (b) urethane dimethacrylate at 1-25 wt. %, (c) hydrogenated rosin, (d) alcohol, and (e) sucralose as a sweetener.

<Embodiment 25> An oral liquid dental suspension including (a) cyclodextrin, (b) xylitol, (c) PAP, (d) sodium cocoyl isethionate, (e) tetrasodium pyrophosphate, (f) nano-hydroxyapatite, (g) PVP, (h) phenoxyethanol, (i) potassium citrate, (j) menthol, (k) ethylhexylglycerin, (1) urethane dimethacrylate, (m) hydrogenated rosin, (n) alcohol, and (o) titanium dioxide.

<Embodiment 26> An oral liquid dental suspension including a tooth whitening agent, a solvent, a resin, wherein upon drying the composition forms a film on tooth surfaces with a thickness under 50 μm.

<Embodiment 27> An oral liquid dental suspension including (a) a tooth whitening agent at 0.1-45 wt. %, (b) a resin and a urethane in a combined amount of 30-60 wt. %, and (c) a remineralization agent at 0.05-10 wt. %.

<Embodiment 28> An oral liquid dental suspension including a tooth whitening agent, a solvent, and a resin, wherein the varnish provides sustained release of the whitening agent for at least 4 hours.

<Embodiment 29> An oral liquid dental suspension including (a) a tooth whitening agent, (b) a solvent, (c) a resin, and (d) a glazing agent, wherein the suspension is air-tight and packaged in single-use blister packs.

<Embodiment 30> An oral liquid dental suspension including (a) peroxide-based whitening agent, (b) resin, and (c) a pH adjusting agent such that the composition has a pH less than 5.

<Embodiment 31> An oral liquid dental suspension including (a) a tooth whitening agent at 0.1-45 wt. %, (b) a resin, (c) a sweetener at 1-10 wt. %, (d) a remineralization agent at 1-10 wt. %, and (e) a fluoride source at 0.01-1 wt. %.

<Embodiment 32> An oral liquid dental suspension including (a) a tooth whitening agent, (b) a solvent, (c) at least one of a resin and a urethane, and (d) flavoring agent present at 0.01-3 wt. %.

<Embodiment 33> An oral liquid dental suspension including (a) a tooth whitening agent that is encapsulated and matrix-bound for sustained release, (b) a resin, and (c) a solvent system that is substantially anhydrous.

<Embodiment 34> An oral liquid dental suspension including (a) a tooth whitening agent, (b) a resin at 10-80 wt. %, (c) a glazing agent at 1-10 wt. %, and (d) shellac at 10-40 wt. %.

<Embodiment 35> An oral liquid dental suspension including (a) a tooth whitening agent, (b) a solvent, and (c) resin, wherein the viscosity of the composition is 200-800 centipoise at room temperature.

<Embodiment 36> An oral liquid dental suspension including (a) a tooth whitening agent at 0.1-45 wt. %, (b) shellac at 10-40 wt. %, and (c) urethane dimethacrylate at 1-25 wt. %.

<Embodiment 37> An oral liquid dental suspension including (a) a tooth whitening agent, (b) a resin, (c) a humectant system including water, glycerin, propylene glycol, xylitol, or sorbitol at 20-70 wt. %.

<Embodiment 38> An oral liquid dental suspension including (a) a tooth whitening agent, (b) a resin, and (c) cyclodextrin at 0.01-2.5 wt. % as a complexing agent.

<Embodiment 39> An oral liquid dental suspension including (a) a tooth whitening agent, (b) a solvent, and (c) a resin, wherein at least one component is selected for controlled release of the whitening agent at a pH between 3 and 7.

<Embodiment 40> An oral liquid dental suspension including (a) a tooth whitening agent, (b) a solvent, (c) a resin, and (d) a polymeric thickener at 0.1-20 wt. %.

<Embodiment 41> An oral liquid dental suspension including (a) a tooth whitening agent at 0.1-45 wt. %, (b) colophonium at 30-60 wt. % as resin, and (c) hydrogenated rosin at 10-30 wt. %.

<Embodiment 42> An oral liquid dental suspension including (a) a bleaching agent encapsulated within nanofibers, (b) a resin, (c) a solvent, and (d) a thickener at 0.5-5 wt. %.

<Embodiment 43> An oral liquid dental suspension including (a) at least two bleaching agents combined, (b) a film-forming resin, and (c) a glazing agent.

<Embodiment 44> An oral liquid dental suspension including (a) phthalimidoperoxycaproic acid (PAP) at 8-12 wt. % encapsulated in a polymeric matrix, (b) shellac at 25-35 wt. %, (c) hydrogenated rosin at 10-25 wt. %, (d) white wax at 0.1-2 wt. %, (e) nano-hydroxyapatite at 0.05-5 wt. %, (f) potassium nitrate at 0.1-5 wt. %, (g) EDTA at 0.05-0.5 wt. %, (h) a humectant at 10-30 wt. %, and (i) with a pH adjusted to 3.5-4.5.

<Embodiment 45> An oral liquid dental suspension including (a) a tooth whitening agent at 0.1-45 wt. %, (b) a film-forming base of both natural and synthetic resin together at a combined amount of 20-60 wt. %, (c) a glazing agent, (d) ethanol as a solvent, and (e) xylitol as a sweetener.

<Embodiment 46> An oral liquid dental suspension including (a) a tooth whitening agent at 0.1-45 wt. %, (b) a resin and a glazing agent, (c) a flavoring agent, and (d) a color-change indicator visible during application.

<Embodiment 47> An oral liquid dental suspension including (a) a peroxide-based tooth whitening agent, (b) a resin, (c) a pH adjusting agent maintaining a pH less than 5, and (d) a polymeric thickener at 0.5-5 wt. %.

<Embodiment 48> An oral liquid dental suspension including (a) a tooth whitening agent at 0.1-45 wt. %, (b) a resin, (c) a sweetener at 1-10 wt. %, (d) a remineralizing agent at 1-10 wt. %, and (e) a fluoride source at 0.01-1 wt. %, wherein the suspension is substantially anhydrous.

<Embodiment 49> An oral liquid dental suspension including (a) a tooth whitening agent, (b) a solvent, (c) a resin, (d) a thickener including ethyl cellulose at 0.05-3 wt. %, and (e) wherein the viscosity of the composition is 300-600 centipoise.

<Embodiment 50> An oral liquid dental suspension including (a) hydrogen peroxide, (b) ethanol, (c) shellac, (d) colophony, (e) mastic, (f) xylitol, (g) sodium saccharin, (h) eucalyptol, (i) thymol, and (j) EDTA.

### Two-Part Oral Liquid Dental Suspension Composition

<Embodiment 51> A two-part oral liquid dental suspension for application to human teeth, including:
a first part including a tooth whitening agent in a liquid vehicle; and
a second part including at least one film-forming resin in a liquid vehicle;
wherein the first and second parts are maintained separate until the time of use, and wherein, upon mixing, the resulting suspension is configured to be applied to teeth and to form a film or varnish on the tooth surface.

<Embodiment 52> A two-part oral liquid dental suspension composition, including:
a first part containing a whitening agent including at least one of hydrogen peroxide, carbamide peroxide, and phthalimidoperoxycaproic acid (PAP); and
a second part containing a film-forming resin including at least one of polyvinyl acetate, shellac, and hydrogenated rosin;
wherein the two parts are provided in separate containers and are mixed prior to application, forming a suspension that dries to a varnish in the presence of saliva.

<Embodiment 53> A two-part oral liquid dental suspension, wherein:
the first part includes 0.1-45 wt. % of a tooth whitening agent in a solvent including at least one of ethanol, water, glycerin, and propylene glycol; and
the second part includes 10-80 wt. % of a resin including at least one of polyvinyl acetate, shellac, colophony, mastic, hydrogenated rosin, and synthetic methacrylate resin, in a solvent including at least one of ethanol, water, glycerin, and propylene glycol.

<Embodiment 54> A two-part oral liquid dental suspension composition including:
a first part including a peroxide- or non-peroxide-based whitening agent; and
a second part including a polymeric or natural resin as a film-former;
wherein the whitening agent and resin are kept isolated until combined for use, and the resulting suspension is non-drip, clump-free, and sets to a continuous film on teeth within 1 minute of application.

<Embodiment 55> A two-part oral liquid dental suspension for teeth whitening, wherein:
the first part includes a tooth whitening agent encapsulated or stabilized in a liquid phase; and
the second part includes a film-former and at least one viscosity modifier;
such that upon mixing, the suspension provides extended contact of the whitening agent with the tooth surface during and after formation of a film.

<Embodiment 56> A two-part oral liquid dental suspension, wherein:
the first part includes hydrogen peroxide at 3-10 wt. % dissolved in ethanol at 10-40 wt. %; and
the second part includes shellac at 25-35 wt. %, hydrogenated rosin at 10-25 wt. %, and a glazing agent at 0.1-2 wt. %;
wherein upon mixing, the suspension forms a varnish having a film thickness of less than 50 μm when dried on teeth.

<Embodiment 57> A two-part oral liquid dental suspension composition, consisting of:
a first part including phthalimidoperoxycaproic acid (PAP) at 8-12 wt. % in a solution further including a stabilizer including at least one of cyclodextrin and EDTA; and
a second part including polyvinyl acetate at 20-50 wt. % and a glazing agent including at least one of white wax, carnauba wax, and microcrystalline wax at 0.1-2 wt. %;
wherein, after mixing, the suspension is applied to teeth to form a varnish with sustained release of PAP.

<Embodiment 58> A two-part oral liquid dental suspension, wherein:
the first part includes a tooth whitening agent and at least one humectant including at least one of glycerin, xylitol, and sorbitol at 5-30 wt. %; and
the second part includes a resin and a remineralizing agent including at least one of nano-hydroxyapatite and tricalcium phosphate at 0.05-5 wt. %;
such that the remineralizing agent becomes dispersed in the final suspension upon mixing.

<Embodiment 59> A two-part oral liquid dental suspension including:
a first part including carbamide peroxide at 10-16 wt. %, a flavoring agent at 0.01-3 wt. %, and a sweetener at 0.01-10 wt. %; and
a second part including shellac and ethyl cellulose at 0.5-5 wt. % as a viscosity modifier;
whereupon mixing, the product forms a varnish that sets in the presence of saliva in less than 60 seconds.

<Embodiment 60> A two-part oral liquid dental suspension, wherein:
the first part contains a whitening agent stabilized with encapsulation in a polymeric or liposomal matrix; and
the second part contains a synthetic resin, a glazing agent, and an adhesion promoter,
wherein, upon combination, the resulting varnish is string-free and maintains the whitening agent in contact with the tooth surface for 0.25-2 hours.

<Embodiment 61> A two-part oral liquid dental suspension, wherein:
the first part includes phthalimidoperoxycaproic acid (PAP) at 8-12 wt. %, potassium citrate at 0.1-5 wt. %, and nano-hydroxyapatite particles at 0.05-5 wt. % with mean particle size less than 100 nm, in a solvent; and
the second part includes shellac at 25-35 wt. %, hydrogenated rosin at 10-25 wt. %, white wax (E901) at 0.1-2 wt. %, and a color-change indicator visible upon application but transparent upon setting.

<Embodiment 62> A two-part oral liquid dental suspension consisting of:
a first part including hydrogen peroxide at 5-15 wt. %, EDTA at 0.05-0.5 wt. %, and xylitol at 1-10 wt. %; and
a second part including shellac, a pH-modifying agent maintaining suspension pH at 3.5-4.5, and a remineralization agent including at least one of nano-hydroxyapatite and tricalcium phosphate.

<Embodiment 63> A two-part oral liquid dental suspension, wherein:
the first part includes a tooth whitening agent, potassium nitrate as a desensitizer, and titanium dioxide at 0.1-1 wt. % as a catalyst or pigment; and
the second part includes polyvinyl acetate, carnauba wax, and a flavoring agent including at least one of menthol, peppermint oil, and eucalyptol at 0.01-3 wt. %.

<Embodiment 64> A two-part oral liquid dental suspension composition for vegan and SLS-free use, wherein:
the first part contains a bleaching agent, a humectant, and no animal-derived or SLS-containing substances; and
the second part contains shellac, synthetic resin, an adhesion promoter, and a glazing agent, all without animal-derived or SLS-containing substances;
wherein, when mixed, the varnish forms a continuous, undetectable film on teeth.

<Embodiment 65> A two-part oral liquid dental suspension, wherein:
the first part includes a whitening agent, potassium nitrate, a remineralization agent, EDTA, a humectant, a sweetener, and a flavoring agent; and
the second part includes shellac, hydrogenated rosin, white wax, ethyl cellulose, an adhesion promoter, a color-change indicator, and a pH-modifying agent;
wherein, upon combination, the final suspension is string-free, non-drip, clump-free, dries in 30 seconds, forms a film with thickness less than 50 μm, and delivers both whitening and remineralization effects within a contact period of at least 0.5 hours.

### Method of Use (Method of Bleaching or Lightening the Color of Human Teeth)

<Embodiment 66> A method of bleaching or lightening the color of human teeth including: applying the oral liquid dental suspension of any one of <Embodiment 1> to <Embodiment 50> or the two-part oral liquid dental suspension of any one of <Embodiment 51> to <Embodiment 65> to the surface of human teeth and allowing the suspension to dry to form a varnish.

<Embodiment 67> A method of bleaching or lightening the color of human teeth including: applying the oral liquid dental suspension of any one of <Embodiment 1> to <Embodiment 50> or the two-part oral liquid dental suspension of any one of <Embodiment 51> to <Embodiment 65> to selected teeth, allowing the suspension to dry to form a varnish, and subsequently allowing contact of the varnish with the teeth for at least 0.1 hour.

<Embodiment 68> A method including: applying the oral liquid dental suspension of any one of <Embodiment 1> to <Embodiment 50> or the two-part oral liquid dental suspension of any one of <Embodiment 51> to <Embodiment 65> to enamel surfaces of human teeth without the use of a tray or mouthguard, and permitting the suspension to dry and adhere as a varnish for a whitening effect.

<Embodiment 69> A method of cosmetic treatment including: topically applying the oral liquid dental suspension of any one of <Embodiment 1> to <Embodiment 50> or the two-part oral liquid dental suspension of any one of <Embodiment 51> to <Embodiment 65> to a human tooth, permitting drying to form a continuous film, and leaving the varnish in contact with the tooth for a period of time.

<Embodiment 70> A method including: applying the oral liquid dental suspension of any one of <Embodiment 1> to <Embodiment 50> or the two-part oral liquid dental suspension of any one of <Embodiment 51> to <Embodiment 65> to the visible surfaces of maxillary and mandibular teeth of a subject, allowing the suspension to set, and then allowing normal oral activity.

<Embodiment 71> A method including: brushing, spraying, swabbing, or painting the oral liquid dental suspension of any one of <Embodiment 1> to <Embodiment 50> or the two-part oral liquid dental suspension of any one of <Embodiment 51> to <Embodiment 65> onto human teeth, allowing it to dry and form a varnish, and maintaining oral hygiene during the whitening period.

<Embodiment 72> A method including: delivering the oral liquid dental suspension of any one of <Embodiment 1> to <Embodiment 50> or the two-part oral liquid dental suspension of any one of <Embodiment 51> to <Embodiment 65> to one or more human teeth by spraying, brushing, or painting, and letting it set to form an adherent varnish for at least 10 minutes.

<Embodiment 73> A method including: applying the oral liquid dental suspension of any one of <Embodiment 1> to <Embodiment 50> or the two-part oral liquid dental suspension of any one of <Embodiment 51> to <Embodiment 65> to the labial or buccal surfaces of teeth, allowing the suspension to dry in the presence of saliva, thereby forming a whitening varnish layer.

<Embodiment 74> A method including: providing the oral liquid dental suspension of any one of <Embodiment 1> to <Embodiment 50> or the two-part oral liquid dental suspension of any one of <Embodiment 51> to <Embodiment 65> to a patient for at-home use, painting the suspension on the teeth, and permitting it to set as a whitening varnish over a desired period.

<Embodiment 75> A method including: applying the oral liquid dental suspension of any one of <Embodiment 1> to <Embodiment 50> or the two-part oral liquid dental suspension of any one of <Embodiment 51> to <Embodiment 65> to human teeth in a dental office, allowing it to dry as a film, and subsequently removing the varnish after a pre-determined contact time.

<Embodiment 76> A method including: cleaning the surface of human teeth; applying the oral liquid dental suspension of any one of <Embodiment 1> to <Embodiment 50> or the two-part oral liquid dental suspension of any one of <Embodiment 51> to <Embodiment 65> to the cleaned teeth; and allowing the suspension to dry to form a whitening varnish.

<Embodiment 77> A method including: drying the labial surfaces of anterior teeth; applying the oral liquid dental suspension of any one of <Embodiment 1> to <Embodiment 50> or the two-part oral liquid dental suspension of any one of <Embodiment 51> to <Embodiment 65> thereto; and allowing it to form a self-adhering varnish within 60 seconds.

<Embodiment 78> A method including: painting the oral liquid dental suspension of any one of <Embodiment 1> to <Embodiment 50> or the two-part oral liquid dental suspension of any one of <Embodiment 51> to <Embodiment 65> onto discolored teeth, forming a varnish by drying in ambient conditions, and repeating application daily for at least three days.

<Embodiment 79> A method including: applying t the oral liquid dental suspension of any one of <Embodiment 1> to <Embodiment 50> or the two-part oral liquid dental suspension of any one of <Embodiment 51> to <Embodiment 65> to the surface of human teeth; exposing the varnish to visible, ultraviolet, or LED light for a predetermined period; and maintaining the varnish on teeth for whitening.

<Embodiment 80> A method including: removing extrinsic stains from teeth using abrasive paste prior to application; applying the oral liquid dental suspension of any one of <Embodiment 1> to <Embodiment 50> or the two-part oral liquid dental suspension of any one of <Embodiment 51> to <Embodiment 65> to tooth surfaces; and letting the varnish adhere for 10-120 minutes.

<Embodiment 81> A method including: applying the oral liquid dental suspension of any one of <Embodiment 1> to <Embodiment 50> or the two-part oral liquid dental suspension of any one of <Embodiment 51> to <Embodiment 65> to teeth with a brush; allowing it to form a film upon contact with saliva; and maintaining the varnish in contact with teeth for at least 1 hour.

<Embodiment 82> A method including: applying the oral liquid dental suspension of any one of <Embodiment 1> to <Embodiment 50> or the two-part oral liquid dental suspension of any one of <Embodiment 51> to <Embodiment 65> to the teeth of an adolescent, allowing it to dry, and permitting the subject to eat or drink after varnish formation.

<Embodiment 83> A method including: applying the oral liquid dental suspension of any one of <Embodiment 1> to <Embodiment 50> or the two-part oral liquid dental suspension of any one of <Embodiment 51> to <Embodiment 65> to buccal tooth surfaces immediately after professional dental cleaning, and maintaining the varnish on the tooth surface for extended whitening.

<Embodiment 84> A method including: applying the oral liquid dental suspension of any one of <Embodiment 1> to <Embodiment 50> or the two-part oral liquid dental suspension of any one of <Embodiment 51> to <Embodiment 65> to selected stained or discolored teeth, allowing the suspension to dry and adhere without contacting gingival tissue.

<Embodiment 85> A method including: painting the oral liquid dental suspension of any one of <Embodiment 1> to <Embodiment 50> or the two-part oral liquid dental suspension of any one of <Embodiment 51> to <Embodiment 65> onto the enamel surfaces of teeth with a pen applicator, drying to form a substantially invisible film, and permitting overnight exposure.

<Embodiment 86> A method including: applying the oral liquid dental suspension of any one of <Embodiment 1> to <Embodiment 50> or the two-part oral liquid dental suspension of any one of <Embodiment 51> to <Embodiment 65> to the enamel surface; setting the varnish in the presence of oral moisture for less than one minute; and allowing whitening to occur.

<Embodiment 87> A method including: positioning the oral liquid dental suspension of any one of <Embodiment 1> to <Embodiment 50> or the two-part oral liquid dental suspension of any one of <Embodiment 51> to <Embodiment 65> on the occlusal and facial tooth surfaces, allowing quick drying to form a non-drip film, and repeating the procedure for a multi-phase whitening process.

<Embodiment 88> A method including: brushing the oral liquid dental suspension of any one of <Embodiment 1> to <Embodiment 50> or the two-part oral liquid dental suspension of any one of <Embodiment 51> to <Embodiment 65> onto the teeth, exposing teeth to visible LED light for at least three minutes, and keeping varnish intact for 0.5-2 hours.

<Embodiment 89> A method including: cleaning the surface of human teeth by flossing and brushing; applying the oral liquid dental suspension of any one of <Embodiment 1> to <Embodiment 50> or the two-part oral liquid dental suspension of any one of <Embodiment 51> to <Embodiment 65>; and letting the suspension dry to form a whitening varnish layer.

<Embodiment 90> A method including: applying the oral liquid dental suspension of any one of <Embodiment 1> to <Embodiment 50> or the two-part oral liquid dental suspension of any one of <Embodiment 51> to <Embodiment 65> to teeth prior to bedtime and allowing the dried varnish to remain in contact with the teeth overnight.

<Embodiment 91> A method including: mechanically removing biofilm from tooth surfaces; applying the oral liquid dental suspension of any one of <Embodiment 1> to <Embodiment 50> or the two-part oral liquid dental suspension of any one of <Embodiment 51> to <Embodiment 65>; and setting the varnish by air-drying for 5-30 seconds.

<Embodiment 92> A method including: applying the oral liquid dental suspension of any one of <Embodiment 1> to <Embodiment 50> or the two-part oral liquid dental suspension of any one of <Embodiment 51> to <Embodiment 65> by means of a pre-dosed applicator to the labial surfaces of teeth and allowing the varnish to set and adhere for extended contact.

<Embodiment 93> A method including: delivering the oral liquid dental suspension of any one of <Embodiment 1> to <Embodiment 50> or the two-part oral liquid dental suspension of any one of <Embodiment 51> to <Embodiment 65> as a spray, permitting it to dry and adhere as a uniform film for sustained whitening action of at least one hour.

<Embodiment 94> A method including: applying the oral liquid dental suspension of any one of <Embodiment 1> to <Embodiment 50> or the two-part oral liquid dental suspension of any one of <Embodiment 51> to <Embodiment 65> to the teeth; subsequently rinsing the mouth with a remineralization agent after a predetermined time; and resuming regular oral hygiene.

<Embodiment 95> A method including: pre-treating teeth with a prophylactic cleaning; applying the oral liquid dental suspension of any one of <Embodiment 1> to <Embodiment 50> or the two-part oral liquid dental suspension of any one of <Embodiment 51> to <Embodiment 65>; and exposing the varnish to directed light energy for up to 15 minutes.

<Embodiment 96> A method including: applying the oral liquid dental suspension of any one of <Embodiment 1> to <Embodiment 50> or the two-part oral liquid dental suspension of any one of <Embodiment 51> to <Embodiment 65> to the surface of a crown, bridge, or restoration; allowing the varnish to dry; and maintaining contact for a period effective for whitening.

<Embodiment 97> A method including: applying the oral liquid dental suspension of any one of <Embodiment 1> to <Embodiment 50> or the two-part oral liquid dental suspension of any one of <Embodiment 51> to <Embodiment 65> to one or more dental implants; allowing the suspension to set to form a varnish; and maintaining oral fluid contact to support sustained whitening.

<Embodiment 98> A method including: immediately prior to application, preparing the oral liquid dental suspension of any one of <Embodiment 1> to <Embodiment 50> or the two-part oral liquid dental suspension of any one of <Embodiment 51> to <Embodiment 65> by mixing separate whitening and base components, applying to teeth, and allowing the varnish to form in situ.

<Embodiment 99> A method including: placing the oral liquid dental suspension of any one of <Embodiment 1> to <Embodiment 50> or the two-part oral liquid dental suspension of any one of <Embodiment 51> to <Embodiment 65> on the buccal surfaces of primary or deciduous teeth, drying for less than 60 seconds, and repeating every six months as a whitening procedure.

<Embodiment 100> A method including: applying the oral liquid dental suspension of any one of <Embodiment 1> to <Embodiment 50> or the two-part oral liquid dental suspension of any one of <Embodiment 51> to <Embodiment 65> with a single-use pen applicator, forming a varnish layer, and waiting at least 30 minutes before removing the varnish by gentle brushing.

<Embodiment 101> A method including: applying the oral liquid dental suspension of any one of <Embodiment 1> to <Embodiment 50> or the two-part oral liquid dental suspension of any one of <Embodiment 51> to <Embodiment 65> to teeth; covering the teeth with a light-transmissive mouthpiece; and illuminating the mouthpiece with LED light to accelerate varnish whitening action.

<Embodiment 102> A method including: applying the oral liquid dental suspension of any one of <Embodiment 1> to <Embodiment 50> or the two-part oral liquid dental suspension of any one of <Embodiment 51> to <Embodiment 65>; measuring tooth shade with a digital shade guide both prior to and after treatment; and documenting shade improvement.

<Embodiment 103> A method including: applying the oral liquid dental suspension of any one of <Embodiment 1> to <Embodiment 50> or the two-part oral liquid dental suspension of any one of <Embodiment 51> to <Embodiment 65> to teeth after air abrasion of tooth surfaces; allowing a varnish to form; and repeating application for targeted whitening of individual teeth.

<Embodiment 104> A method including: placing the oral liquid dental suspension of any one of <Embodiment 1> to <Embodiment 50> or the two-part oral liquid dental suspension of any one of <Embodiment 51> to <Embodiment 65> on tooth enamel; allowing the varnish to dry for under 45 seconds; and subjecting the varnish to LED light for at least 5 minutes to catalyze whitening.

<Embodiment 105> A method including: applying the oral liquid dental suspension of any one of <Embodiment 1> to <Embodiment 50> or the two-part oral liquid dental suspension of any one of <Embodiment 51> to <Embodiment 65> to the tooth surfaces of patients with a known gag reflex; allowing the varnish to set without the need for trays or inserts for comfortable home use.

<Embodiment 106> A method including: brushing the oral liquid dental suspension of any one of <Embodiment 1> to <Embodiment 50> or the two-part oral liquid dental suspension of any one of <Embodiment 51> to <Embodiment 65> onto stained areas of artificial veneers, crowns, or bridges; allowing it to form a whitening varnish effective for lightening stains without damaging restorations.

<Embodiment 107> A method including: applying the oral liquid dental suspension of any one of <Embodiment 1> to <Embodiment 50> or the two-part oral liquid dental suspension of any one of <Embodiment 51> to <Embodiment 65> to cleaned teeth; exposing the varnish to UV or halogen light for 3-10 minutes; and rinsing the mouth with water after whitening treatment.

<Embodiment 108> A method including: applying the oral liquid dental suspension of any one of <Embodiment 1> to <Embodiment 50> or the two-part oral liquid dental suspension of any one of <Embodiment 51> to <Embodiment 65> to teeth following use of a mouth rinse containing a bleach activator, and drying the varnish under ambient conditions for less than 30 seconds.

<Embodiment 109> A method including: applying the oral liquid dental suspension of any one of <Embodiment 1> to <Embodiment 50> or the two-part oral liquid dental suspension of any one of <Embodiment 51> to <Embodiment 65> to dental surfaces; setting with LED light for under 10 minutes; and then brushing off the varnish with a soft toothbrush.

<Embodiment 110> A method including: applying the oral liquid dental suspension of any one of <Embodiment 1> to <Embodiment 50> or the two-part oral liquid dental suspension of any one of <Embodiment 51> to <Embodiment 65> to both natural and artificial teeth in the same dental arch; forming a continuous whitening film; and maintaining contact for 1-4 hours.

<Embodiment 111> A method including: sequentially applying the oral liquid dental suspension of any one of <Embodiment 1> to <Embodiment 50> or the two-part oral liquid dental suspension of any one of <Embodiment 51> to <Embodiment 65> to anterior teeth every day for at least 7 days, allowing the varnish to remain overnight during each treatment.

<Embodiment 112> A method including: applying the oral liquid dental suspension of any one of <Embodiment 1> to <Embodiment 50> or the two-part oral liquid dental suspension of any one of <Embodiment 51> to <Embodiment 65> after patient-specific tooth color determination; forming a transparent varnish; and repeating application as needed to achieve a desired shade value.

<Embodiment 113> A method including: applying the oral liquid dental suspension of any one of <Embodiment 1> to <Embodiment 50> or the two-part oral liquid dental suspension of any one of <Embodiment 51> to <Embodiment 65> to the teeth; measuring pH at the varnish-enamel interface to ensure a range between 4.0-5.0 throughout the contact period.

<Embodiment 114> A method including: painting the oral liquid dental suspension of any one of <Embodiment 1> to <Embodiment 50> or the two-part oral liquid dental suspension of any one of <Embodiment 51> to <Embodiment 65> on teeth cleaned with toothpaste including abrasive particles and allowing the varnish to dry to a film of under 50 μm thickness.

<Embodiment 115> A method including: applying the oral liquid dental suspension of any one of <Embodiment 1> to <Embodiment 50> or the two-part oral liquid dental suspension of any one of <Embodiment 51> to <Embodiment 65> to teeth following pretreatment with a biofilm-removal instrument, setting with ambient air in under 30 seconds, and maintaining the whitening varnish until removed by eating or oral hygiene.

<Embodiment 116> A method of bleaching or lightening the color of human teeth, the method including:
(a) providing a two-part oral liquid dental suspension, wherein a first part includes a tooth whitening agent and a second part includes a film-forming resin;
(b) mixing the first and second parts prior to use to form a ready-to-apply suspension;
(c) applying the resulting suspension to the surface of human teeth;
(d) allowing the suspension to dry to form a varnish in situ on the teeth;
wherein the varnish adheres to the teeth for an effective period to enable bleaching.

<Embodiment 117> A method of whitening human teeth including the steps of:
(a) preparing a two-phase liquid dental suspension, wherein a whitening agent is present in a first phase and a film-forming resin is present in a second phase;
(b) sequentially or simultaneously combining the two phases immediately before application;
(c) applying the combined mixture directly to buccal and/or labial tooth surfaces;
(d) allowing the mixture to dry in the presence of saliva to form an adherent varnish;
wherein the dried varnish enables extended contact between the whitening agent and tooth enamel.

<Embodiment 118> A method of lightening the color of human teeth, including:
providing a two-part kit having (i) a peroxide-based whitening solution and (ii) a resin/solvent matrix;
mixing the solutions to form an oral dental suspension;
painting the combined suspension onto selected teeth;
and allowing the suspension to set at ambient oral conditions to form a continuous film that remains adherent for at least 15 minutes.

<Embodiment 119> A method including:
(a) removing surface biofilm from teeth using a prophylactic cleaning step;
(b) providing a two-compartment applicator, one compartment containing a whitening agent and the other a resin base;
(c) actuating or mixing contents to produce a single active dental suspension;
(d) applying the suspension to tooth surfaces;
(e) subjecting the applied suspension to visible LED light for between 3 and 15 minutes;
wherein a whitening effect is realized and less than 25 mg of whitening agent is ingested or washed away.

<Embodiment 120> A method for whitening teeth, including:
providing a dual-phase composition, wherein the tooth whitening agent and resin are separated until use;
mixing both phases in a dispensing pen prior to application;
depositing the mixed composition onto anterior teeth;
allowing the suspension to dry in situ to form a non-drip, continuous varnish film; and
subsequently optionally rinsing the mouth after up to 1 hour.

<Embodiment 121> A method of treating human teeth for color lightening including:
(a) dispensing a two-part oral liquid dental suspension in which the tooth whitening agent is encapsulated in a polymeric phase and the resin base is liquid;
(b) combining the two parts immediately before use;
(c) applying the resultant composition to the enamel of human teeth;
(d) permitting the formulation to dry within 60 seconds into an adhesive film that slowly releases the whitening agent.

<Embodiment 122> A method of dental bleaching, including:
(a) mixing a peroxide-containing solution and a resin-containing solution to yield a homogeneous oral suspension immediately before use;
(b) delivering the suspension to at least one tooth using a brush, swab, or similar applicator;
(c) exposing the coated tooth surface to light energy (including at least one of UV, halogen, LED, and plasma arc) for up to 20 minutes post-application;
wherein the composition forms a varnish adhering for at least 0.5 hours and the procedure results in visible tooth whitening.

<Embodiment 123> A method including:
(a) cleaning the teeth and drying the surfaces to be treated;
(b) combining a bleaching-agent solution and a film-forming solution (kept separate until use) to generate an oral liquid dental suspension;
(c) applying the freshly mixed suspension to teeth surfaces;
(d) allowing the suspension to set and form a varnish with a thickness less than 50 μm that remains in contact with tooth surfaces for at least 30 minutes.

<Embodiment 124> A method for lightening teeth, including:
providing a two-part oral liquid dental suspension including, in a first component, a whitening agent and, in a second component, a hydrophobic resin base;
mixing both components immediately prior to administration;
covering all or selected teeth with the resultant mixture;
allowing the mixture to dry and adhere persistently for between 0.25 and 2 hours while the patient is able to eat or drink.

<Embodiment 125> A method of bleaching human teeth, the method including:
(a) providing a dual-syringe system with a first syringe containing a stabilized tooth whitening agent and a second syringe containing a resinous film former;
(b) expelling both syringes simultaneously into a mixing chamber immediately before use;
(c) applying the freshly mixed suspension to the outer surfaces of the teeth by brush;
(d) maintaining the coating for a treatment period of 10 to 60 minutes, after which the mouth is optionally rinsed with a remineralizing mouth rinse.

### Kit

<Embodiment 126> A kit including the oral liquid dental suspension of any one of <Embodiment 1> to <Embodiment 50> and an applicator.

<Embodiment 127> A kit including the two-part oral liquid dental suspension of any one of <Embodiment 51> to <Embodiment 65> and an applicator.

<Embodiment 128> A kit including the oral liquid dental suspension of any one of <Embodiment 1> to <Embodiment 50> and packaging configured for single-use dosing.

<Embodiment 129> A kit including the two-part oral liquid dental suspension of any one of <Embodiment 51> to <Embodiment 65> and packaging adapted for mixing prior to use.

<Embodiment 130> A kit including the oral liquid dental suspension of any one of <Embodiment 1> to <Embodiment 50> and instructions for use, wherein the instructions detail application to human teeth.

<Embodiment 131> A kit including the oral liquid dental suspension of any one of <Embodiment 1> to <Embodiment 50>, an applicator brush, and a mouthpiece for delivering light energy.

<Embodiment 132> A kit including the two-part oral liquid dental suspension of any one of <Embodiment 51> to <Embodiment 65>, a dual-chamber syringe, and an LED light mouthpiece.

<Embodiment 133> A kit including the oral liquid dental suspension of any one of <Embodiment 1> to <Embodiment 50>, a pen applicator, and a digital tooth shade guide.

<Embodiment 134> A kit including the two-part oral liquid dental suspension of any one of <Embodiment 51> to <Embodiment 65>, a power supply, and a portable charging base.

<Embodiment 135> A kit including the oral liquid dental suspension of any one of <Embodiment 1> to <Embodiment 50>, an applicator brush, and a bleaching light source selected from UV, halogen, LED, or plasma arc.

<Embodiment 136> A kit including the two-part oral liquid dental suspension of any one of <Embodiment 51> to <Embodiment 65>, an applicator, and a carrying case suitable for home and clinical use.

<Embodiment 137> A kit including the oral liquid dental suspension of any one of <Embodiment 1> to <Embodiment 50>, an LED mouthpiece, and a smartphone application for tracking usage.

<Embodiment 138> A kit including the two-part oral liquid dental suspension of any one of <Embodiment 51> to <Embodiment 65>, a digital shade measuring device, and pre-measured ampoules.

<Embodiment 139> A kit including the oral liquid dental suspension of any one of <Embodiment 1> to <Embodiment 50>, a user manual, a storage case, and a power adapter.

<Embodiment 140> A kit including the two-part oral liquid dental suspension of any one of <Embodiment 51> to <Embodiment 65>, a single-use applicator, and a heat-generating mouthpiece.

<Embodiment 141> A kit including the oral liquid dental suspension of any one of <Embodiment 1> to <Embodiment 50>, a pre-treatment remineralization rinse, and an applicator.

<Embodiment 142> A kit including the two-part oral liquid dental suspension of any one of <Embodiment 51> to <Embodiment 65>, a post-treatment desensitizing rinse, and an applicator.

<Embodiment 143> A kit including the oral liquid dental suspension of any one of <Embodiment 1> to <Embodiment 50>, a shade guide for evaluating whitening results, and a pen applicator.

<Embodiment 144> A kit including the two-part oral liquid dental suspension of any one of <Embodiment 51> to <Embodiment 65>, a blister pack, and a brush applicator.

<Embodiment 145> A kit including the oral liquid dental suspension of any one of <Embodiment 1> to <Embodiment 50>, a pre-treatment oral rinse containing a bleach activator, and an applicator.

<Embodiment 146> A kit including the two-part oral liquid dental suspension of any one of <Embodiment 51> to <Embodiment 65>, a dual-syringe applicator, and instructions indicating sequential use.

<Embodiment 147> A kit including the oral liquid dental suspension of any one of <Embodiment 1> to <Embodiment 50>, a multi-compartment applicator, and a digital timer.

<Embodiment 148> A kit including the two-part oral liquid dental suspension of any one of <Embodiment 51> to <Embodiment 65>, an autoclavable mouthpiece, and a manual for professional use.

<Embodiment 149> A kit including the oral liquid dental suspension of any one of <Embodiment 1> to <Embodiment 50>, a portable charging case, and a mouthpiece for light-induced activation.

<Embodiment 150> A kit including the two-part oral liquid dental suspension of any one of <Embodiment 51> to <Embodiment 65>, an LED light device, a controller, and a shade evaluation card.

<Embodiment 151> A kit including the oral liquid dental suspension of any one of <Embodiment 1> to <Embodiment 50>, a pre-measured pen applicator, a pre-treatment teeth desensitizer rinse, and a carrying case.

<Embodiment 152> A kit including the two-part oral liquid dental suspension of any one of <Embodiment 51> to <Embodiment 65>, a dual-barrel syringe, a silicone-tipped applicator, and a post-bleaching remineralization mouth rinse.

<Embodiment 153> A kit including the oral liquid dental suspension of any one of <Embodiment 1> to <Embodiment 50>, a digital tooth shade guide, a user manual, an LED-illuminated mouthpiece, and a portable charging base.

<Embodiment 154> A kit including the two-part oral liquid dental suspension of any one of <Embodiment 51> to <Embodiment 65>, a temperature-controlled LED mouthpiece, a lanyard, and an instruction booklet.

<Embodiment 155> A kit including the oral liquid dental suspension of any one of <Embodiment 1> to <Embodiment 50>, a smartphone-enabled mouthpiece, a tint-changing shade guide, and a USB power adapter.

<Embodiment 156> A kit including the two-part oral liquid dental suspension of any one of <Embodiment 51> to <Embodiment 65>, a precision dosing applicator, an LED light tray, and a wireless sensor to track usage.

<Embodiment 157> A kit including the oral liquid dental suspension of any one of <Embodiment 1> to <Embodiment 50>, a prepackaged oral rinse with a bleach activator, a heat-activated mouthpiece, and instructions for light and heat application.

<Embodiment 158> A kit including the two-part oral liquid dental suspension of any one of <Embodiment 51> to <Embodiment 65>, a separate container for each part, a mixing chamber, and a brush tip for direct application.

<Embodiment 159> A kit including the oral liquid dental suspension of any one of <Embodiment 1> to <Embodiment 50>, a shade-measuring app, a removable controller, a timer, and a hygiene storage box.

<Embodiment 160> A kit including the two-part oral liquid dental suspension of any one of <Embodiment 51> to <Embodiment 65>, a modular applicator system, refill containers, and a UV-C sterilizable mouthpiece.

<Embodiment 161> A kit including the oral liquid dental suspension of any one of <Embodiment 1> to <Embodiment 50>, a removable tray, a flexible pen applicator, a digital shade comparator, and a charger.

<Embodiment 162> A kit including the two-part oral liquid dental suspension of any one of <Embodiment 51> to <Embodiment 65>, a rechargeable mouthpiece, an app-controlled timer, and a multi-language instruction guide.

<Embodiment 163> A kit including the oral liquid dental suspension of any one of <Embodiment 1> to <Embodiment 50>, a combination applicator (brush and spray), a user-identification card, and a shade analysis mat.

<Embodiment 164> A kit including the two-part oral liquid dental suspension of any one of <Embodiment 51> to <Embodiment 65>, unit-dose pouches, a brush-head applicator, and a waterproof protective box.

<Embodiment 165> A kit including the oral liquid dental suspension of any one of <Embodiment 1> to <Embodiment 50>, an LED-embedded mouth tray, a magnifying mirror, a carrying case, and a shade-matching reference card.

<Embodiment 166> A kit including the two-part oral liquid dental suspension of any one of <Embodiment 51> to <Embodiment 65>, a digital tracking module, a universal USB-C charger, and a fast-mix automatic applicator.

<Embodiment 167> A kit including the oral liquid dental suspension of any one of <Embodiment 1> to <Embodiment 50>, a quick-drying tray, a visual application indicator, and a single-use ampoule.

<Embodiment 168> A kit including the two-part oral liquid dental suspension of any one of <Embodiment 51> to <Embodiment 65>, a personalized user profile app, a snap-fit carrying case, and single-use light-pulse mouthpiece.

<Embodiment 169> A kit including the oral liquid dental suspension of any one of <Embodiment 1> to <Embodiment 50>, a dual-tip applicator, a brushing timer, a digital instruction panel, and single-use shade swatches.

<Embodiment 170> A kit including the two-part oral liquid dental suspension of any one of <Embodiment 51> to <Embodiment 65>, a wireless charging stand, a removable LED array, and a hygiene compartment.

<Embodiment 171> A kit including the oral liquid dental suspension of any one of <Embodiment 1> to <Embodiment 50>, a foldable power charger, augmented reality installation software, and a video instruction module.

<Embodiment 172> A kit including the two-part oral liquid dental suspension of any one of <Embodiment 51> to <Embodiment 65>, a consumable cartridge system, color-coded component holders, and Bluetooth connectivity for usage tracking.

<Embodiment 173> A kit including the oral liquid dental suspension of any one of <Embodiment 1> to <Embodiment 50>, an adjustable mouthpiece, a temperature monitoring device, and a USB stick with digital guides.

<Embodiment 174> A kit including the two-part oral liquid dental suspension of any one of <Embodiment 51> to <Embodiment 65>, a tooth-surface camera for before and after imaging, and a lockable storage vault.

<Embodiment 175> A kit including the oral liquid dental suspension of any one of <Embodiment 1> to <Embodiment 50>, a customizable color-guide, an antimicrobial mouth tray, a travel bag, and a digital progress tracker.

Each issued patent, patent publication, and non-patent literature cited herein is hereby incorporated by reference in its entirety, as if each was incorporated by reference individually.

The invention as set forth in the claims can now be illustrated by the following non-limiting examples.

### EXAMPLES

### Example 1. Dental Varnish (Prophetic) for Teeth Bleaching

### Formulation 1

Ingredients: tooth whitening agent, solvent, sweetener, flavoring agent, and resin.

### Formulation 2

Ingredients: tooth whitening agent, solvent, sweetener, flavoring agent, resin, and urethane.

### Formulation 3

Ingredients: tooth whitening agent, solvent, sweetener, flavoring agent, and urethane.

### Formulation 4

Ingredients: tooth whitening agent, solvent, sweetener, flavoring agent, resin, and teeth desensitizer.

### Formulation 5

Ingredients: tooth whitening agent, solvent, sweetener, flavoring agent, resin, urethane, and teeth desensitizer.

### Formulation 6

Ingredients: tooth whitening agent, solvent, sweetener, flavoring agent, urethane, and teeth desensitizer.

### Formulation 7

Ingredients: tooth whitening agent, solvent, sweetener, flavoring agent, resin, and sequestering agent.

### Formulation 8

Ingredients: tooth whitening agent, solvent, sweetener, flavoring agent, resin, urethane, and sequestering agent.

### Formulation 9

Ingredients: tooth whitening agent, solvent, sweetener, flavoring agent, glazing agent, urethane, and sequestering agent.

### Formulation 10

Ingredients: tooth whitening agent, solvent, sweetener, flavoring agent, resin, and fluoridation agent.

### Formulation 11

Ingredients: tooth whitening agent, solvent, sweetener, flavoring agent, resin, urethane, and fluoridation agent.

### Formulation 12

Ingredients: tooth whitening agent, solvent, sweetener, flavoring agent, urethane, and fluoridation agent.

### Formulation 13

Ingredients: Ethanol, Hydrogen Peroxide, White Wax (E901), Shellac (E904), Colophony, Mastic, Saccharin (E954), and Raspberry Essence (including Ethyl Butyrate, Geraniol, Iris Resinoid, Isoamyl Acetate, Jasmine Absolute, Vanillin, and Propylene Glycol).

### Formulation 14

Ingredients: Ethanol, Carbamide Peroxide, White wax (E901), Shellac (E904), Colophony, Mastic, Saccharin (E954), and Raspberry Essence (which contains Ethyl Butyrate, Geraniol, Iris Resinoid, Isoamyl Acetate, Jasmine Absolute, Vanillin and Propylene Glycol).

### Formulation 15

Ingredients: Ethanol, Hydrogen Peroxide, White wax (E901), Shellac (E904), Colophony, Mastic, Saccharin (E954), Ethylenediaminetetraacetic acid (EDTA), and Raspberry Essence (which contains Ethyl Butyrate, Geraniol, Iris Resinoid, Isoamyl Acetate, Jasmine Absolute, Vanillin and Propylene Glycol).

### Formulation 16

Ingredients: Ethanol, Carbamide Peroxide, White wax (E901), Shellac (E904), Colophony, Mastic, Saccharin (E954), Ethylenediaminetetraacetic acid (EDTA), and Raspberry Essence (which contains Ethyl Butyrate, Geraniol, Iris Resinoid, Isoamyl Acetate, Jasmine Absolute, Vanillin and Propylene Glycol).

### Formulation 17

Ingredients: Ethanol, Hydrogen Peroxide, White wax (E901), Shellac (E904), Colophony, Mastic, Saccharin (E954), Eucalyptol, and Thymol.

### Formulation 18

Ingredients: Ethanol, Carbamide Peroxide, White wax (E901), Shellac (E904), Colophony, Mastic, Saccharin (E954), Eucalyptol, and Thymol.

### Formulation 19

Ingredients: Ethanol, Hydrogen Peroxide, White wax (E901), Shellac (E904), Colophony, Mastic, Saccharin (E954), Ethylenediaminetetraacetic Acid (EDTA), Eucalyptol, and Thymol.

### Formulation 20

Ingredients: Ethanol, Carbamide Peroxide, White wax (E901), Shellac (E904), Colophony, Mastic, Saccharin (E954), Ethylenediaminetetraacetic Acid (EDTA), Eucalyptol, and Thymol.

### Formulation 21

Ingredients: Ethanol, Hydrogen Peroxide, White wax (E901), Shellac (E904), Colophony, Mastic, Xylitol, Sodium Saccharin, and Raspberry Essence (which contains Ethyl Butyrate, Geraniol, Iris Resinoid, Isoamyl Acetate, Jasmine Absolute, Vanillin and Propylene Glycol).

### Formulation 22

Ingredients: Ethanol, Carbamide Peroxide, White wax (E901), Shellac (E904), Colophony, Mastic, Xylitol, Sodium Saccharin, and Raspberry Essence (which contains Ethyl Butyrate, Geraniol, Iris Resinoid, Isoamyl Acetate, Jasmine Absolute, Vanillin and Propylene Glycol).

### Formulation 23

Ingredients: Ethanol, Hydrogen Peroxide, White wax (E901), Shellac (E904), Colophony, Mastic, Xylitol, Sodium Saccharin, Ethylenediaminetetraacetic acid (EDTA), and Raspberry Essence (which contains Ethyl Butyrate, Geraniol, Iris Resinoid, Isoamyl Acetate, Jasmine Absolute, Vanillin and Propylene Glycol).

### Formulation 24

Ingredients: Ethanol, Carbamide Peroxide, White wax (E901), Shellac (E904), Colophony, Mastic, Xylitol, Sodium Saccharin, Ethylenediaminetetraacetic acid (EDTA), and Raspberry Essence (which contains Ethyl Butyrate, Geraniol, Iris Resinoid, Isoamyl Acetate, Jasmine Absolute, Vanillin and Propylene Glycol).

### Formulation 25

Ingredients: Ethanol, Hydrogen Peroxide, White wax (E901), Shellac (E904), Colophony, Mastic, Xylitol, Sodium Saccharin, and Thymol.

### Formulation 26

Ingredients: Ethanol, Carbamide Peroxide, White wax (E901), Shellac (E904), Colophony, Mastic, Xylitol, Sodium Saccharin, Eucalyptol, and Thymol.

### Formulation 27

Ingredients: Ethanol, Hydrogen Peroxide, White wax (E901), Shellac (E904), Colophony, Mastic, Xylitol, Sodium Saccharin, Ethylenediaminetetraacetic Acid (EDTA), Eucalyptol, and Thymol.

### Formulation 28

Ingredients: Ethanol, Carbamide Peroxide, White wax (E901), Shellac (E904), Colophony, Mastic, Xylitol, Sodium Saccharin, Ethylenediaminetetraacetic Acid (EDTA), Eucalyptol, and Thymol.

### Formulation 29

Ingredients: Hydrogen Peroxide, Urethane Dimethacrylate, Hydrogenated Rosin, Alcohol, Natural and Artificial flavors, Titanium Dioxide, Sucralose.

### Formulation 30

Ingredients: Carbamide Peroxide, Urethane Dimethacrylate, Hydrogenated Rosin, Alcohol, Natural and Artificial flavors, Titanium Dioxide, Sucralose.

### Formulation 31

Ingredients: Cyclodextrin, Xylitol, Phthalimidoperoxycaproic Acid (PAP), Sodium Cocoyl Isethionate, Tetrasodium Pyrophosphate, Hydroxyapatite (nano), Mica (CI77019), PVP, Phenoxyethanol, Potassium Citrate, Sucralose, Menthol, Monosodium Citrate, Ethylhexylglycerin, Urethane Dimethacrylate, Hydrogenated Rosin, Alcohol, Natural and Artificial flavors, Titanium Dioxide, Sucralose.

### Formulation 32

Ingredients: Hydrogenated Rosin, Ethanol, Phthalimidoperoxycaproic Acid (PAP), Sweetener, Flavoring Agent.

### Example 2. LED Teeth Whitening Equipment

### Kit 1

Equipment: Autoclavable Illuminate Heat Mouthpiece & Case, Power Control with Built-in Timer, Portable Charging Base, Power Adapter & USB Cable, User Manual.

### Kit 2

Equipment: Mouthpiece & Silicone Cover; Controller with temperature indicator, countdown timer, and extended battery life; Lanyard for hands-free whitening, USB Cord, User Manual.

### Kit 3

Equipment: Mouthpiece (hands-free, smartphone-powered LED accelerator light) with USB cable, storage case.

### Kit 4

Equipment: Mouthpiece (rechargeable LED accelerator light), storage and charging case, charging cable.

### Kit 5

Equipment: USB-powdered LED mouthpiece.

### Kit 6

Equipment: A fixed-shape mouth tray with LED lights; Adaptors to fit USB, USB Type-C and lightning (iPhone) sockets; A shade guide; Instruction manual.

### Kit 7

Equipment: Mouthpiece (LED—405 nm violet light—which is illuminated by 32 LEDs).

Additional teeth whitening equipment suitable for use in the present invention can be found, e.g., at U.S. Pat. Nos. D636,074; 8,029,278; 8,371,853; 8,591,227; 9,271,638; D765,255; 9,572,645; 795,501; 9,827,174; 10,123,860; D888,961; 8,215,954; 8,262,306; 8,262,390; 8,398,324; 8,419,306; 8,591,227; 9,572,645; D795,501; 10,201,408; 8,075,875; 10,231,811; U.S. application Ser. No. 29/644,369; U.S. application Ser. No. 29/696,510; and U.S. application Ser. No. 16/593,104.

### Example 3. Remineralization Rinse, Pre-Bleaching and/or Post Bleaching

### Oral Rinse 1

Ingredients: Water, Clorostan® (stabilized chlorine dioxide), trisodium phosphate, citric acid, flavor, sucralose.

### Oral Rinse 2

Ingredients: Potassium Nitrate, Sodium Fluoride, Disodium Phosphate, FD&C Blue #1, Flavor, Glycerin, Methylparaben, PEG-60 Hydrogenated Castor Oil, Poloxamer 407, Propylparaben, Sodium Benzoate, Sodium Phosphate, Sodium Saccharin, Sorbitol, Water.

### Oral Rinse 3

Ingredients: Purified Water, Xylitol, Erythritol, Vegetable Glycerin, Echinacea Purpurea, Chamomile, Olive Leaf, Thyme, Aloe Vera, Calcium Glycerophosphate, Cocamidopropyl Betaine, Honeysuckle, Neem Leaf, Sage, Myrrh Gum Resin, Cranberry, Licorice Root, Potassium Nitrate, CoQ10, Ascorbic Acid, Tea Tree Oil, Oat Beta Glucan, Natural Mint Flavors, Natural Plant Colors.

### Example 4. Post Bleaching Fluoridation Rinse

### Oral Rinse 1

Ingredients: Sodium fluoride, Water, glycerin, flavor, cetylpyridinium chloride, sodium saccharin, phosphoric acid, sodium benzoate, sucralose, poloxamer 407, benzoic acid, disodium phosphate, propylene glycol, blue 1.

### Oral Rinse 2

Ingredients: Sodium fluoride, menthol, water, vegetable glycerin, polysorbate 80, flavor, xylitol, erythritol, poloxamer 407, sodium benzoate, tea tree oil.

### Oral Rinse 3

Ingredients: Sodium Fluoride, Water, Glycerin, Propylene Glycol, Sorbitol, Poloxamer 338, Poloxamer 407, Flavor, PEG-40 Castor Oil, Phosphoric Acid, Sodium Saccharin, Cetylpyridinium Chloride, Disodium Phosphate, Potassium Sorbate, Sucralose, FD&C Green 3.

### Oral Rinse 4

Ingredients: Sodium Fluoride, Water, Glycerin, Propylene Glycol, Sorbitol, Poloxamer 407, Poloxamer 338, Flavor, PEG-40 Hydrogenated Castor Oil, Phosphoric Acid, Sodium Saccharin, Cetylpyridinium Chloride, Disodium Phosphate, Potassium Sorbate, Sucralose, FD&C Green 3.

### Oral Rinse 5

Ingredients: Sodium Fluoride, water, glycerin, flavor, cetylpyridinium chloride, sodium saccharin, phosphoric acid, methylparaben, sucralose, poloxamer 407, propylparaben, disodium phosphate, blue 1.

### Example 5. Dental Bleaching Agents

Selection of bleaching agent can depend on several factors. First, dental professionals typically recommend a few main bleaching agents for teeth whitening:

### Hydrogen Peroxide

Hydrogen peroxide is one of the most commonly used and effective teeth whitening agents. It's a powerful bleaching compound that can provide fast results. Professional in-office treatments typically use hydrogen peroxide in concentrations ranging from 15 wt. % to 43 wt. %.

### Carbamide Peroxide

Carbamide peroxide is another widely used bleaching agent. It breaks down into hydrogen peroxide and urea when applied to the teeth. Professional take-home whitening kits often contain carbamide peroxide in concentrations between 10 wt. % to 35 wt. %.

### Key Differences

Concentration: In-office treatments use higher concentrations of these agents compared to at-home products. Professional in-office systems may contain up to 43 wt. % peroxide, while at-home systems typically range from 3 wt. % to 20 wt. %.

Application Time: Hydrogen peroxide works more quickly but for a shorter duration. Carbamide peroxide releases hydrogen peroxide more slowly, allowing for longer application times.

Effectiveness: Both agents are effective, but the higher concentrations used by dental professionals often provide faster and more dramatic results compared to over-the-counter products.

Dental professionals carefully select the appropriate bleaching agent and concentration based on factors like the patient's tooth sensitivity, desired results, and treatment method (in-office vs take-home). The controlled application of these agents in a professional setting helps ensure safe and effective teeth whitening outcomes.

Second, in addition to the commonly used hydrogen peroxide and carbamide peroxide, there are a few other bleaching agents that have been explored for teeth whitening:

Phthalimidoperoxycaproic Acid (PAP+)

PAP+ is a newer, non-peroxide-based whitening agent:
It's an organic peroxy acid that can effectively whiten tooth enamel.
Unlike peroxide-based products, PAP+ doesn't produce reactive oxygen species or free radicals during the oxidation process, which may reduce tooth sensitivity.
The PAP+ formula often includes nano-hydroxyapatite to prevent mineral loss and potassium citrate to help desensitize exposed dentin.

### Sodium Perborate

While less common, sodium perborate has been used in some whitening applications:
It's a milder bleaching agent compared to hydrogen peroxide.
When mixed with water, it forms hydrogen peroxide, making it an indirect source of the bleaching agent.

### Example 6. Use of Dental Varnish (Prophetic) for Teeth Bleaching

Dental bleaching varnishes are topical treatments applied to teeth to help whiten the tooth/teeth and optionally to strengthen enamel. Key points about cleaching varnishes used in dental treatments:

### Composition

Dental varnishes typically contain:
Bleaching agent (peroxide) as the active ingredient
A resin or synthetic base that allows the varnish to adhere to tooth surfaces
Flavoring agents to improve taste

### Application Process

The varnish is painted directly onto clean, dry teeth using an applicator (small brush)·
It sets quickly when it comes in contact with saliva.
The sticky coating forms a thin layer over the teeth
Application takes only a few minutes per patient

### Mechanism of Action

The bleaching agent in the varnish is slowly released over time as the varnish breaks down.
This allows prolonged contact between the bleaching agent and tooth enamel
The remineralization rinse helps strengthen enamel and makes teeth more resistant to acid attacks that cause decay

### Usage Guidelines

Typically applied multiple times during the year, at home or in dental offices.
Can be used on both natural teeth as well as implants, veneers, etc.
Safe for use in adolescents younger than 18 years old
Patients can eat or drink after application.
Convenient for use at bedtime, to sleep with the varnish on the teeth
Can be selectively used with one or more teeth

### Benefits

More effective than other dental bleaching treatments due to prolonged contact time.
Can be used with (or without) LED Teeth Whitening Equipment
Easy and quick to apply, with minimal risk of ingestion
Bleaching varnish can be a safe and effective way to deliver bleaching agent(s). Its ability to adhere to teeth and release bleaching agent over time makes it an important tool in teeth whitening.

### Example 7. Dental Varnish: Resin and/or Synthetic Base

Dental bleaching varnishes will typically use one of two main types of bases that allow them to adhere to tooth surfaces:

### 1. Colophony (Rosin) Base:

Colophony is a natural resin derived from pine trees.
It forms a sticky film that adheres well to tooth surfaces when applied

### 2. Synthetic Resin Base:

Made of synthetic polymers rather than natural resins
Examples include shellac and methacrylate-based resins
Designed to improve on some limitations of colophony-based varnishes
   Key Properties of these Bases that Allow Adherence:
They form a sticky film when applied to dry tooth surfaces
The film hardens quickly when exposed to saliva
The hardened film adheres strongly to enamel for several hours
The film slowly breaks down over time, releasing bleaching agent

### Some Key Differences:

Synthetic bases like shellac can form a thinner film than colophony, potentially allowing better bleaching agent release
Synthetic bases may be less allergenic for patients with pine/tree nut allergies
Some synthetic bases are designed to be more comfortable or less noticeable for patients.
The base allows the varnish to stay in place on the teeth long enough for the bleaching agent to be absorbed into the enamel, enhancing its effectiveness as a cavity prevention treatment. The choice of base impacts properties like adherence time, comfort, and bleaching agent release rate.

### Example 7. Formulation Challenges and Solutions

There are several formulation challenges in developing a dental varnish that contains a bleaching agent for tooth whitening. These formulation challenges make it difficult to develop an effective and safe dental varnish that combines both protective and whitening properties. As illustrated below, the optimal solution to each formulation challenge may involve combining multiple strategies based on the specific formulation requirements.

| Formulation Challenges | Solution |
|---|---|
| Stability of the bleaching agent: | 1. Use of stabilizers: |
| Many common bleaching | Incorporating stabilizers into the formulation can help |
| agents like hydrogen peroxide | prevent decomposition of the bleaching agent. Some |
| are unstable and can degrade | options include: |
| over time, especially when | pH control using acids like phosphoric acid or citric |
| exposed to light or heat. | acid |
| Maintaining the stability and | Addition of metal ion sequestrants like EDTA |
| efficacy of the bleaching agent | Use of antioxidants, though these have some |
| in the varnish formulation is | limitations |
| challenging. | 2. Anhydrous or low-water formulations: |
| | Reducing water content helps prevent hydrolysis |
| | and decomposition of peroxide-based bleaching |
| | agents. Options include: |
| | Using glycerol or other non-aqueous solvents as the |
| | base |
| | Developing dry powder formulations that are |
| | reconstituted before use |
| | Creating anhydrous gels or strips |
| | 3. Encapsulation or controlled release systems: |
| | Protecting the bleaching agent within a carrier system can |
| | enhance stability. Examples include: |
| | Loading carbamide peroxide into electrospun |
| | nanofiber films |
| | Encapsulating the bleaching agent in microspheres |
| | Using polymer matrices to control release and |
| | exposure to moisture |
| | These approaches help maintain the stability and efficacy of |
| | bleaching agents like hydrogen peroxide and carbamide |
| | peroxide by limiting their exposure to factors that promote |
| | decomposition, such as water, heat, light, and contaminants. |
| | The optimal solution may involve combining multiple |
| | strategies based on the specific formulation requirements. |
| Compatibility with varnish | 1. Use of anhydrous or low-water formulations: |
| base: The bleaching agent needs | Reducing water content helps prevent hydrolysis |
| to be compatible with the resin | and decomposition of peroxide-based bleaching |
| or synthetic base used in dental | agents. |
| varnishes. Many bleaching | Options include using glycerol or other non-aqueous |
| agents are water-based, while | solvents as the base, developing dry powder |
| varnish bases are often | formulations that are reconstituted before use, or |
| hydrophobic, making it difficult | creating anhydrous gels or strips. |
| to incorporate the bleaching | 2. Encapsulation or controlled release systems: |
| agent uniformly. | Protecting the bleaching agent within a carrier |
| | system can enhance stability and compatibility. |
| | Examples include loading the bleaching agent into |
| | electrospun nanofiber films, encapsulating it in |
| | microspheres, or using polymer matrices to control |
| | release and exposure to moisture. |
| | 3. Modification of the varnish base: |
| | Using synthetic resin bases instead of traditional |
| | colophony (rosin) can improve compatibility with |
| | bleaching agents. |
| | Synthetic bases like shellac and methacrylate-based |
| | resins are designed to improve on some limitations |
| | of colophony-based varnishes. |
| | These synthetic bases can form a thinner film than |
| | colophony, potentially allowing better release of |
| | active ingredients like bleaching agents. |
| | These approaches help address the compatibility issues |
| | between bleaching agents and varnish bases by either |
| | modifying the bleaching agent formulation, protecting it |
| | from direct contact with the base, or altering the base itself |
| | to be more compatible with the active ingredients. The |
| | optimal solution may involve combining multiple strategies |
| | based on the specific formulation requirements. |
| Adhesion and retention: The | 1. Use of adhesives: |
| varnish needs to adhere well to | Applying an adhesive prior to sealant placement |
| tooth surfaces and stay in place | significantly improves retention. |
| long enough for the bleaching | An etch-and-rinse adhesive system is recommended |
| agent to be effective. However, | over self-etch adhesives for better long-term |
| the presence of a bleaching | effectiveness. |
| agent may interfere with the | Adhesives help prevent microleakage around |
| adhesion properties of the | sealants in addition to improving retention. |
| varnish base. | 2. Surface preparation techniques: |
| | Air abrasion, polishing, and bur preparation are |
| | more effective than bristle brushing for preparing |
| | the tooth surface. |
| | These preparation techniques should be used in |
| | combination with acid etching for best results. |
| | Enamoplasty (mechanical preparation with a bur) |
| | prior to sealant placement reduces leakage and |
| | improves retention compared to less invasive |
| | techniques. |
| | 3. Optimized varnish formulation: |
| | Adding citric acid (1-3 wt. %) to the varnish |
| | formulation can enhance retention by slightly |
| | etching sound enamel surfaces. |
| | Using an adhesion-promoting agent in the varnish |
| | formula, as done with Enamelast ™ fluoride varnish, |
| | can improve adhesion. |
| | Maintaining a non-aqueous mixture in the varnish, |
| | allowing better interaction with tooth surfaces upon |
| | application. |
| | These approaches focus on improving the tooth surface |
| | preparation, using adhesive systems, and optimizing the |
| | varnish formulation to enhance adhesion and retention of |
| | dental varnishes and sealants. The optimal solution may |
| | involve combining multiple strategies based on the specific |
| | formulation requirements. |
| Controlled release: The | 1. Encapsulation in micro/nanocapsules: |
| formulation needs to allow for | Active ingredients can be physically encapsulated |
| controlled release of the | inside aqueous pockets of micro/nanocapsules. |
| bleaching agent over time to | This allows for controlled release as the capsule |
| achieve whitening effects | walls break down over time. |
| without causing tooth | Liposomal formulations are particularly promising |
| sensitivity or gum irritation. | due to their biocompatibility. |
| | 2. Physical adsorption via electronic interactions: |
| | Active ingredients can be physically adsorbed onto |
| | polymer particles through electronic interactions. |
| | This provides a way to control release through the |
| | strength of the electrostatic bonds. |
| | Can be effective for small hydrophilic molecules |
| | that are difficult to encapsulate. |
| | 3. Covalent conjugation: |
| | Active ingredients can be chemically conjugated to |
| | polymer backbones via covalent bonds. |
| | This allows for very controlled release as the bonds |
| | break down. |
| | Can enable high and stable loading of hydrophilic |
| | drugs in systems like micelles. |
| | Key advantages of these approaches: |
| | Allow for sustained release over time rather than |
| | immediate burst release |
| | Can protect unstable active ingredients from |
| | degradation |
| | Enable incorporation of hydrophilic molecules into |
| | hydrophobic carriers |
| | Provide ways to control the release kinetics through |
| | formulation |
| | The optimal approach depends on the specific active |
| | ingredient properties, desired release profile, and delivery |
| | system. Often a combination of strategies may be used to |
| | achieve the desired controlled release characteristics. |
| pH balance: Many bleaching | 1. Use of acidifying components: |
| agents work best at specific pH | Adding organic or inorganic acids like citric acid, |
| levels. Maintaining the optimal | phosphoric acid, or malic acid to lower the pH of the |
| pH for bleaching while ensuring | varnish. |
| the varnish remains safe for oral | The acid can be added to adjust the pH to less than |
| use is challenging. | 5, preferably less than 4.5, and even down to about |
| | 3. |
| | This helps stabilize the bleaching agent and improve |
| | adhesion to tooth surfaces. |
| | 2. Anhydrous or low-water formulations: |
| | Developing dry powder formulations that are |
| | reconstituted before use. |
| | Creating anhydrous gels or using non-aqueous |
| | solvents like glycerol as the base. |
| | Reducing water content helps prevent hydrolysis |
| | and decomposition of peroxide-based bleaching |
| | agents. |
| | 3. Encapsulation or controlled release systems: |
| | Protecting the bleaching agent within a carrier |
| | system like microspheres or polymer matrices. |
| | This allows for controlled release and exposure to |
| | moisture, helping maintain optimal pH for the |
| | bleaching agent. |
| | Encapsulation can also help separate incompatible |
| | components like fluoride and calcium until |
| | application. |
| | These approaches help maintain the stability of bleaching |
| | agents while allowing for an optimal pH that balances |
| | efficacy and safety when applied to teeth. The specific |
| | solution may depend on the particular formulation and |
| | desired properties of the varnish. |
| Viscosity: The addition of a | 1. Use of anhydrous or low-water formulations: |
| bleaching agent may alter the | Reducing water content helps prevent hydrolysis |
| viscosity of the varnish, | and decomposition of peroxide-based bleaching |
| potentially affecting its | agents. |
| application properties and | Options include using glycerol or other non-aqueous |
| ability to form a uniform | solvents as the base, developing dry powder |
| coating on teeth. | formulations that are reconstituted before use, or |
| | creating anhydrous gels or strips. |
| | This approach helps maintain stability of the |
| | bleaching agent while allowing for proper viscosity. |
| | 2. Optimizing shellac concentration: |
| | Shellac is commonly used as a film-forming agent in |
| | dental varnishes. |
| | Embodiments having between 25-35 wt. % shellac |
| | (by weight) have exhibited desirable viscosity |
| | properties. |
| | Too high shellac content creates a thick and |
| | noticeable coating, while too low content allows |
| | undesired migration. |
| | A varnish of about 30 wt. % shellac is found to |
| | provide sufficient viscosity to inhibit undesired |
| | varnish migration while still allowing proper flow |
| | and application. |
| | 3. Use of viscosity modifiers: |
| | Adding viscosity modifiers like ethyl cellulose can |
| | help achieve the desired rheological properties. |
| | Lower viscosity ethyl cellulose can be used to create |
| | higher overall viscosity compositions compared to |
| | previous formulations. |
| | This allows for a substantially uniform suspension |
| | of the bleaching agent without phase separation. |
| | Target viscosity ranges of 200-800 centipoise, |
| | preferably 300-600 centipoise, have been found |
| | effective for dental varnish applications. |
| | These approaches allow for optimizing the viscosity to |
| | achieve proper flow, adhesion, and stability characteristics |
| | while incorporating bleaching agents into dental varnish |
| | formulations. |
| Enamel safety: The formulation | 1. Use of remineralizing agents: |
| must ensure that the bleaching | Incorporating remineralizing agents like calcium |
| process does not cause | phosphate compounds (e.g. CPP-ACP, ACP) into |
| excessive demineralization or | the varnish formulation can help protect and |
| damage to the tooth enamel. | strengthen enamel. |
| | These agents provide calcium and phosphate ions |
| | that can help remineralize enamel and counteract |
| | potential demineralization from bleaching agents. |
| | For example, MI Varnish containing CPP-ACP has |
| | shown increased enamel microhardness compared to |
| | fluoride-only varnishes. |
| | 2. Controlled fluoride release: |
| | Formulating the varnish to allow controlled, |
| | sustained release of fluoride can help protect |
| | enamel. |
| | Adding compounds like monobasic ammonium |
| | phosphate (MAP) has been shown to increase |
| | fluoride release in aqueous environments while |
| | maintaining varnish stability. |
| | Controlled fluoride release helps strengthen enamel |
| | and resist acid attacks during and after bleaching. |
| | 3. Optimized pH balance: |
| | Maintaining an optimal pH balance in the varnish |
| | formulation is crucial for enamel safety. |
| | Adding mild acids like citric acid (1-3 wt. %) can |
| | slightly etch sound enamel to improve varnish |
| | adhesion without causing damage. |
| | Controlling pH helps prevent excessive enamel |
| | demineralization while allowing the bleaching agent |
| | to remain effective. |
| | These approaches focus on protecting and strengthening the |
| | enamel through remineralization, controlled fluoride |
| | delivery, and optimized pH, helping to mitigate potential |
| | negative effects of bleaching agents on tooth enamel. The |
| | optimal solution may involve combining multiple strategies |
| | based on the specific formulation requirements. |
| Taste and comfort: | 1. Use of flavoring agents and sweeteners: |
| Incorporating a bleaching agent | Adding flavoring components like mint, bubble |
| while maintaining acceptable | gum, or fruit flavors to mask any unpleasant taste. |
| taste and comfort for the patient | Incorporating sweeteners such as xylitol, saccharin, |
| can be difficult. | or sucralose to improve palatability. |
| | For example, Enamelast ™ fluoride varnish offers |
| | flavors like Cool Mint, Orange Cream, Bubble Gum, |
| | Walterberry, and Caramel Cream. |
| | 2. Optimizing viscosity and texture: |
| | Developing formulations with smooth textures that |
| | feel clean and undetectable on teeth. |
| | Aiming for viscosities around 200-800 centipoise to |
| | allow proper flow and application while preventing |
| | grittiness. |
| | Using synthetic resin bases like shellac instead of |
| | traditional colophony to create thinner, less |
| | noticeable films. |
| | 3. Anhydrous or low-water formulations: |
| | Reducing water content to prevent hydrolysis and |
| | decomposition of bleaching agents. |
| | Using non-aqueous solvents like glycerol as the |
| | base. |
| | This approach helps maintain stability of the |
| | bleaching agent while improving texture and |
| | reducing grittiness. |
| | These solutions focus on improving flavor, optimizing |
| | texture/viscosity, and using anhydrous formulations to |
| | enhance the taste and comfort of dental varnishes |
| | containing bleaching agents. One goal is to create a product |
| | that patients find pleasant and are more likely to tolerate for |
| | the necessary treatment duration. The optimal solution may |
| | involve combining multiple strategies based on the specific |
| | formulation requirements. |
| Regulatory compliance: | Based on the search results and regulatory considerations, |
| Developing a varnish with both | here are three key solutions to address regulatory |
| fluoride (for cavity prevention) | compliance for a dental varnish containing a bleaching |
| and a bleaching agent may face | agent for teeth whitening: |
| regulatory challenges, as it | 1. Develop a two-part system: |
| would combine a medical | Create a separate fluoride varnish component and |
| device (fluoride varnish) with a | bleaching agent component that are mixed or |
| cosmetic product (tooth | applied sequentially at the time of use. |
| whitener). | This avoids potential conflicts between fluoride |
| | varnishes regulated as medical devices (under 21 |
| | CFR 872.3260) and bleaching agents typically |
| | regulated as cosmetics. |
| | The fluoride varnish component can be positioned as |
| | a cavity varnish device, while the bleaching |
| | component is treated as a separate cosmetic product. |
| | 2. Focus on cavity varnish indications: |
| | Formulate and market the product primarily as a |
| | cavity varnish under 21 CFR 872.3260. |
| | Avoid making caries prevention claims, which are |
| | considered drug claims under FDA regulations. |
| | Emphasize indications like occluding dentin tube for |
| | hypersensitivity relief, which align with the cavity |
| | varnish device classification. |
| | Position any whitening effects as secondary to the |
| | primary cavity varnish function. |
| | 3. Conduct comprehensive testing and documentation: |
| | Perform biocompatibility testing according to ISO |
| | 10993-1 standards. |
| | Conduct stability testing to demonstrate shelf life of |
| | the combined formulation. |
| | Document testing for properties like tubule |
| | occlusion, viscosity, retention, fluoride release, and |
| | whitening efficacy. |
| | Prepare a thorough 510(k) submission with all |
| | required performance data to demonstrate |
| | substantial equivalence to legally marketed predicate |
| | devices. |
| | Include detailed information on the novel |
| | combination of fluoride varnish and bleaching agent |
| | to address any potential safety or efficacy concerns. |
| | These approaches aim to navigate the regulatory landscape |
| | by clearly defining the product category, avoiding |
| | conflicting claims, and providing robust data to support |
| | safety and efficacy as a dental device with whitening |
| | capabilities. Careful formulation, positioning, and |
| | documentation can help address the regulatory challenges of |
| | combining a fluoride varnish with a bleaching agent. |
| Shelf life: Ensuring a | 1. Anhydrous or low-water formulations: |
| reasonable shelf life for the | Reducing water content helps prevent hydrolysis |
| product while maintaining the | and decomposition of peroxide-based bleaching |
| efficacy of both the varnish | agents. |
| base and the bleaching agent is | Options include using glycerol or other non-aqueous |
| a significant challenge. | solvents as the base, developing dry powder |
| | formulations that are reconstituted before use, or |
| | creating anhydrous gels. |
| | This approach helps maintain stability of the |
| | bleaching agent over time. |
| | 2. Use of stabilizers: |
| | Incorporating stabilizers into the formulation can |
| | help prevent decomposition of the bleaching agent. |
| | Options include pH control using acids like |
| | phosphoric acid or citric acid. |
| | Addition of metal ion sequestrants like EDTA. |
| | Use of antioxidants, though these have some |
| | limitations. |
| | Stabilizers can help extend shelf life by preventing |
| | degradation of active ingredients. |
| | 3. Optimized packaging: |
| | Using opaque, air-tight packaging that minimizes |
| | exposure to light, heat, and moisture. |
| | Single-dose packaging, like unit-dose blister packs, |
| | can help maintain stability by preventing repeated |
| | exposure to air and contaminants. |
| | Proper storage instructions, such as keeping the |
| | product at controlled temperatures, may be crucial. |
| | These approaches help maintain the stability and efficacy of |
| | bleaching agents by limiting their exposure to factors that |
| | promote decomposition, such as water, heat, light, and |
| | contaminants. The optimal solution may involve combining |
| | multiple strategies based on the specific formulation |
| | requirements. Proper packaging and storage conditions are |
| | also crucial for maximizing shelf life of dental varnishes |
| | containing bleaching agents. |

1. A method of bleaching or lightening the color of human teeth, the method comprising sequentially performing steps (1)-(4) without any intervening treatment:
(1) removing biofilm and extrinsic staining present on the teeth employing at least one of: (a) a slow-speed rotary handpiece and a rubber cup with abrasive paste, (b) an instrument which emits a powder, water, and compressed air, (c) toothpaste comprising abrasive particles, (d) an ultrasonic scaler, hand instruments, and/or a prophy paste;
(2) rinsing the mouth with an oral liquid solution comprising a bleach activator comprising tetraacetylethylenediamine (TAED), sodium nonanoyloxybenzenesulfonate (NOBS), or a combination thereof;
(3) applying an oral liquid dental suspension to the surface of human teeth and allowing the suspension to dry to form a varnish, the suspension comprising: (a) tooth whitening agent; (b) solvent; and (c) at least one of (i) resin and (ii) urethane;
(4) subjecting the varnish to light energy for 0.05-0.25 hours, the light energy comprising at least one of UV, halogen, LED, and plasma arc;
wherein,
upon applying the oral liquid dental suspension to the surface of human teeth, the oral liquid dental suspension dries to form the varnish within 30 seconds; and
the varnish effectively adheres to teeth's surface, thereby allowing the tooth whitening agent to stay in contact with the teeth for 0.1-12 hours.

2. The method of claim 1, further comprising before the oral liquid dental suspension is applied to the human teeth, applying a titanium oxide (TiO₂) nanoparticle-based catalyst to the surface of the teeth.

3. The method of claim 1, further comprising after the varnish is formed, subjecting the varnish to light energy and/or heat, which is intended to accelerate the process of bleaching.

4. The method of claim 1, further comprising after the varnish is formed, subjecting the varnish to light energy comprising UV, halogen, LED, or plasma arc.

5. The method of claim 1, further comprising after the varnish is formed, subjecting the varnish to light energy which excites the tooth whitening agent molecules without overheating the pulp of the teeth.

6. The method of claim 1, further comprising after the varnish is formed, subjecting the varnish to light energy for 0.05-0.25 hours.

7. The method of claim 1, further comprising rinsing the mouth with an oral liquid solution 0.1-1.5 hours after the oral liquid dental suspension is applied to the human teeth, wherein the oral liquid solution comprises a teeth desensitizer or remineralization agent comprising 0.1-5 wt. % potassium nitrate, 0.1-5 wt. % potassium citrate, 0.05-5 wt. % tricalcium phosphate, 0.05-5 wt. % tetrapotassium pyrophosphate, 0.05-10 wt. % hydroxyapatite, or a combination thereof.

8. The method of claim 1, wherein the tooth whitening agent comprises phthalimidoperoxycaproic acid (PAP) present at 1-25 wt. % of the oral liquid dental suspension.

9. The method of claim 1, wherein the oral liquid dental suspension includes nano-hydroxyapatite particles at 0.05-10 wt. % as a remineralization agent and potassium nitrate as a desensitizing agent at 0.1-5 wt. %.

10. The method of claim 1, wherein the oral liquid dental suspension comprises hydrogen peroxide as the whitening agent at 2-45 wt. %.

11. The method of claim 1, wherein the oral liquid dental suspension comprises carbamide peroxide as the whitening agent at 10-44 wt. %.

12. The method of claim 1, wherein the oral liquid dental suspension includes ethylenediaminetetraacetic acid (EDTA) as a stabilizer at 0.01-2.5 wt. %.

13. The method of claim 1, wherein the oral liquid dental suspension comprises xylitol as a sweetener at 1-10 wt. %, sucralose as a sweetener at 0.01-1 wt. %, or a combination thereof.

14. The method of claim 1, wherein the oral liquid dental suspension comprises a glazing agent comprising at least one of beeswax, carnauba wax, and microcrystalline wax, each present at 0.1-10 wt. %.

15. The method of claim 1, wherein the oral liquid dental suspension comprises a flavoring agent comprising at least one of peppermint oil, spearmint, menthol, thymol, eucalyptol, and fruit flavor, at 0.01-3 wt. %.

16. The method of claim 1, wherein the oral liquid dental suspension comprises titanium dioxide as a catalyst or pigment at 0.1-5 wt. %.

17. The method of claim 1, wherein the oral liquid dental suspension comprises a synthetic resin, comprising at least one of polyvinyl acetate and methacrylate polymer, at 20-60 wt. %.

18. The method of claim 1, wherein the oral liquid dental suspension comprises shellac at 10-40 wt. %.

19. The method of claim 1, wherein the oral liquid dental suspension comprises urethane dimethacrylate at 1-25 wt. %.

20. The method of claim 1, wherein the oral liquid dental suspension comprises sodium fluoride as a fluoridation agent at 0.01-1 wt. %.

21. The method of claim 1, wherein the oral liquid dental suspension comprises cyclodextrin as a complexing agent at 0.01-2.5 wt. %.

22. The method of claim 1, wherein the oral liquid dental suspension comprises glycerin as a humectant at 10-30 wt. %.

23. The method of claim 1, wherein the oral liquid dental suspension comprises ethanol as solvent at 5-40 wt. %.

24. The method of claim 1, wherein the oral liquid dental suspension comprises propylene glycol as solvent at 2-12 wt. %.

25. The method of claim 1, wherein the oral liquid dental suspension comprises nano-hydroxyapatite particles at 0.05-5 wt. %, with an average particle size less than 100 nm.

26. The method of claim 1, wherein the oral liquid dental suspension comprises a glazing agent comprising at least one of white wax (E901), candelilla wax, and shellac, each present at 0.1-10 wt. %.

27. The method of claim 1, wherein the oral liquid dental suspension further comprises potassium citrate at 0.1-5 wt. % as a desensitizing or remineralizing agent, tricalcium phosphate as a remineralization agent at 0.05-5 wt. %, or a combination thereof.

28. The method of claim 1, wherein the oral liquid dental suspension comprises a thickener comprising ethyl cellulose at 0.05-3 wt. %.

29. A method of bleaching or lightening the color of human teeth, the method comprising sequentially performing steps (1) (5) without intervening treatment:
(1) removing biofilm and extrinsic staining present on the teeth employing at least one of: (a) a slow-speed rotary handpiece and a rubber cup with abrasive paste, (b) an instrument which emits a powder, water, and compressed air, (c) toothpaste comprising abrasive particles, and (d) an ultrasonic scaler, hand instruments, and/or a prophy paste;
(2) rinsing the mouth with an oral liquid solution comprising a bleach activator comprising tetraacetylethylenediamine (TAED), sodium nonanoyloxybenzenesulfonate (NOBS), or a combination thereof;
(3) applying an oral liquid dental suspension to the surface of human teeth and allowing the suspension to dry to form a varnish within 30 seconds, the suspension comprising:
(a) a tooth whitening agent comprising at least one of phthalimidoperoxycaproic acid (PAP) at 1-12 wt. %, and hydrogen peroxide at 3-10 wt. %;
(b) a synthetic resin base comprising shellac at 25-35 wt. % and hydrogenated rosin at 10-25 wt. %;
(c) at least one glazing agent comprising at least one of white wax (E901) and carnauba wax, at 0.1-2 wt. %;
(d) nano-hydroxyapatite particles at 0.05-5 wt. % as a remineralization agent with an average particle size of 20-80 nm;
(e) a desensitizing agent comprising at least one of potassium nitrate at 0.1-5 wt. % and potassium citrate at 0.1-5 wt. %;
(f) a stabilizer, ethylenediaminetetraacetic acid (EDTA), at 0.05-0.5 wt. %;
(g) a humectant comprising at least one of glycerin at 10-30 wt. % and propylene glycol at 10-30 wt. %;
(h) a pH adjusting agent maintaining the pH of the suspension between 3.5 and 4.5;
(i) and being free of animal-derived ingredients and sodium lauryl sulfate;
(4) subjecting the varnish to visible LED light energy for 0.1-0.15 hours, without exceeding 45° C. at the tooth surface;
(5) rinsing the mouth 0.25-2.5 hours after varnish application with an oral liquid solution comprising at least one of a teeth desensitizer and remineralization agent;
wherein the varnish is non-drip, string-free, clump-free, and forms a continuous film with thickness less than 50 μm that remains essentially intact for 0.25-2.5 hours, and wherein less than 25 mg of tooth whitening agent is ingested or washed away in a single session.

30. The method of claim 29, wherein:
(a) the tooth whitening agent comprises phthalimidoperoxycaproic acid (PAP) at 8-12 wt. % and is encapsulated in a polymeric matrix for controlled release;
(b) the suspension further comprises a flavoring agent comprising at least one of menthol and eucalyptol, at 0.1-0.5 wt. %, a sweetener comprising at least one of xylitol and sucralose, at 1-3 wt. %, and a color-change indicator that is visible during application but becomes transparent when fully set;
(c) the dental suspension is provided in a pre-measured single-dose pen applicator or blister pack; and
(d) the varnish is additionally exposed to a titanium oxide (TiO₂) nanoparticle-based catalyst (anatase phase, 0.1-1 wt. %) applied to the teeth prior to suspension application.
